(19) United States
(12) Patent Application Publication
(10) Pub. No.: US 2022/0040166 A1
(43) Pub. Date: Feb. 10, 2022

(54) Methods and Compositions of Treating an Ophthalmic Condition

(71) Applicant: KARTOS THERAPEUTICS, New York, NY (US)

(72) Inventor: Wayne Rothbaum, Delray Beach, FL (US)

(21) Appl. No.: 17/413,188

(22) PCT Filed: Dec. 11, 2019
(86) PCT No.: PCT/US19/65732

§ 371 Date: Jun. 11, 2021

Related U.S. Application Data
(60) Provisional application No. 62/777,905, filed on Dec. 11, 2018.

(51) Int. Cl. A61K 31/451; A61K 9/51; A61P 27/02

(52) U.S. Cl. CPC A61K 31/451; A61P 27/02; A61K 9/5153

(57) ABSTRACT

Therapeutic methods and pharmaceutical compositions for treating an ophthalmic condition including age-related macular degeneration in a human subject are described. In certain embodiments, the invention includes therapeutic methods using an MDM2 inhibitor.

### FIELD OF THE INVENTION

Methods and compositions of treating an ophthalmic condition including age-related macular degeneration (AMD) using a Mouse double minute 2 homolog (MDM2) inhibitor are disclosed herein.

### BACKGROUND OF THE INVENTION

p53 is a tumor suppressor and transcription factor that responds to cellular stress by activating the transcription of numerous genes involved in cell cycle arrest, apoptosis, senescence, and DNA repair. Unlike normal cells, which have infrequent cause for p53 activation, tumor cells are under constant cellular stress from various insults including hypoxia and pro-apoptotic oncogene activation. Thus, there is a strong selective advantage for inactivation of the p53 pathway in tumors, and it has been proposed that eliminating p53 function may be a prerequisite for tumor survival. In support of this notion, three groups of investigators have used mouse models to demonstrate that absence of p53 function is a continuous requirement for the maintenance of established tumors. When the investigators restored p53 function to tumors with inactivated p53, the tumors regressed.

Further, p53 is reported to participate in the regulation of angiogenesis. Abnormal retinal vascular proliferation is implicated in ophthalmic conditions such as age-related macular degeneration (AMD), geographic atrophy, proliferative diabetic retinopathy, and retinopathy of prematurity. One common treatment for such proliferative diseases is the use of neutralizing antibodies or soluble receptors to neutralize vascular endothelial growth factor-A (VEGF-A) in the eye. However, not all patients exhibit response to anti-VEGF directed therapeutics, including engineered antibodies, soluble receptor fusion proteins, ankyrin-repeat proteins and dual-action agents that target VEGF-A isoforms plus additional soluble factors. Among those who initially respond, a high percentage of patients will have progressive loss of visual acuity after starting a VEGF-directed therapy.

The present invention relates to methods of treating an ophthalmic condition in a human subject with an MDM2 inhibitor, alone or in combination with one or more additional pharmaceutically active agents. It is noted that human MDM2 can also be referred to as HDM2 or hMDM2.

### SUMMARY OF THE INVENTION

In one aspect, the present application relates to a pharmaceutical composition formulated for opthalmological administration comprising an MDM2 inhibitor, and a pharmaceutically acceptable excipient, wherein the MDM2 inhibitor is a compound of Formula (I)

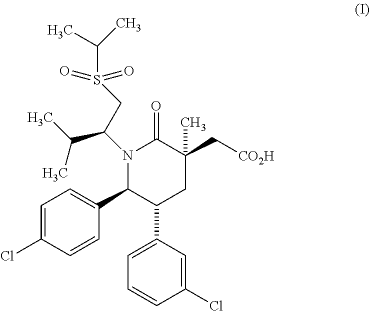 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically acceptable salt thereof.

In one aspect, the present application relates to a pharmaceutical composition comprising nanoparticles comprising an MDM2 inhibitor, a surfactant, and a pharmaceutically acceptable excipient, wherein the MDM2 inhibitor is a compound of Formula (I)

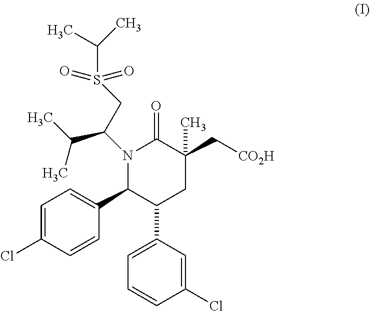 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically acceptable salt thereof.

In an embodiment, the nanoparticles further comprise a polymer.

In an embodiment, the nanoparticles have a median particle size less than 150 nm.

In an embodiment, the nanoparticles have PDI less than 0.2.

In an embodiment, the MDM2 inhibitor is encapsulated in the nanoparticles.

In an embodiment, the nanoparticles comprise a polymer selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In an embodiment, the surfactant is selected from the group consisting of polysorbatee, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

In an embodiment, the surfactant is tocopheryl polyethylene glycol succinate (TPGS).

In an embodiment, wherein the polymer is poly(lactic co-glycolic acid) (PLGA).

In an embodiment, PLGA has an average molecular weight of about 10 kDa, 30 kDa, or 100 kDa.

In an embodiment, PLGA has lactic acid/glycolic acid ratio of 50:50 or 75:25.

In an embodiment, the nanoparticles further comprise a hydrogel.

In an embodiment, the hydrogel is selected from the group consisting of poly(propylene oxide), poly(ethylene oxide), poloxamers (pluronics), chitosan, gelatin, cellulose derivatives, glycol chitin, poly(N-isopropylacrylamide (PNIPAAm), PEG-PLGA-PEG, [poly(D, L-lactide)-poly(ethyleneglycol)-poly(D,L-lactide) (PDLLA-PEG-PDLLA), and combinations thereof.

In another aspect, the present invention relates to a method of treating an ophthalmic condition in a human subject in need thereof selected from the group consisting of wet age-related macular degeneration, dry age-related macular degeneration, diabetic retinopathy (DR), neovascular age-related macular degeneration (nAMD), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, ocular inflammation, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, the method comprising the step of administering to a human subject in need thereof the pharmaceutical composition comprising the nanoparticles described herein by intravitreal injection.

In an embodiment, the ophthalmic condition is neovascular age-related macular degeneration (nAMD) or dry age-related macular degeneration.

In an embodiment, the ophthalmic condition is geographic atrophy (GA).

In an embodiment, the geographic atrophy (GA) is multilobular geographic atrophy or unilobular geographic atrophy.

In an embodiment, the ophthalmic condition is macular edema following retinal vein occlusion (RVO).

In an embodiment, the ophthalmic condition is diabetic macular edema (DME).

In an embodiment, the ophthalmic condition is diabetic retinopathy (DR).

In an embodiment, the ophthalmic condition is inflammatory/infectious retinal neovascularization/edema.

In an embodiment, the human is treated daily, twice a week, three time a week, weekly, biweekly or monthly.

In another aspect, the present invention relates to a method of treating an ophthalmic condition in a human subject in need thereof selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, the method comprising the step of administering to a human subject in need thereof a therapeutically effective amount of an MDM2 inhibitor, wherein the MDM2 inhibitor is a compound of Formula (I) or a compound of Formula (II):

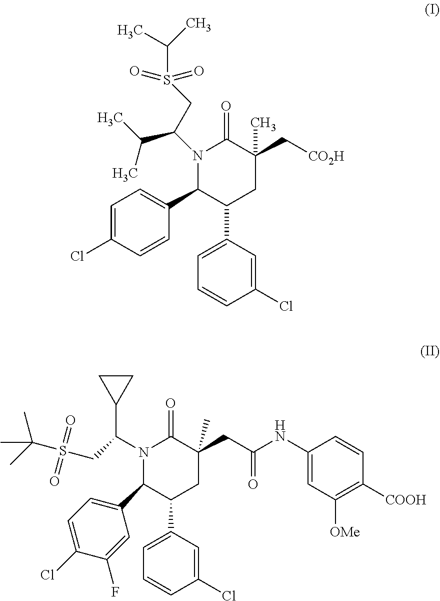 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In an embodiment, the ophthalmic condition is wet age-related macular degeneration.

In an embodiment, the ophthalmic condition is dry age-related macular degeneration.

In an embodiment, the ophthalmic condition is geographic atrophy (GA). In an embodiment, the geographic atrophy (GA) is multilobular geographic atrophy or unilobular geographic atrophy.

In an embodiment, the ophthalmic condition is macular edema following retinal vein occlusion (RVO).

In an embodiment, the ophthalmic condition is diabetic macular edema (DME).

In an embodiment, the ophthalmic condition is diabetic retinopathy (DR).

In an embodiment, the ophthalmic condition is inflammatory/infectious retinal neovascularization/edema.

In an embodiment, the compound of Formula (I) or Formula (II) is in a crystalline form.

In an embodiment, the compound of Formula (I) or Formula (II) is in a free form.

In an embodiment, the MDM2 inhibitor is a pharmaceutically acceptable salt of a compound of Formula (I) or Formula (II).

In an embodiment, the compound of Formula (I) or Formula (II) is in an amorphous form.

In an embodiment, the crystalline form of Formula (I) is characterized by a powder X-ray diffraction pattern comprising at least three peaks at diffraction angle 2 theta degrees selected from a group consisting of peaks at approximately 11.6, 12.4, 18.6, 19.0, 21.6 and 23.6±0.1.

In an embodiment, the human is treated with the MDM2 inhibitor on days 1-7 of 21-day cycle, wherein on days 8-21 the human is not treated with the MDM2 inhibitor.

In an embodiment, the compound of Formula (I) or Formula (II) is orally administered.

In an embodiment, the compound of Formula (I) or Formula (II) is topically administered.

In an embodiment, the compound of Formula (I) or Formula (II) is in a dosage form. The dosage form is a solution, suspension, ointment, gel, hydrogel, drug delivery device, tablet, or capsule.

A MDM2 inhibitor for use in treating an ophthalmic condition in a human subject in need thereof selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, wherein the MDM2 inhibitor is a compound of Formula (I) or Formula (II) or a pharmaceutically acceptable salt thereof.

### BRIEF DESCRIPTION OF THE DRAWINGS

The foregoing summary, as well as the following detailed description of the invention, will be better understood when read in conjunction with the appended drawings.

FIG. 1 illustrates the vitreous humor concentration of the compound Formula (I) (“Comp-A”) after intravitreal injection of a solution of Comp-A. The solution of Comp-A refers to the solution of Comp-A in PBS at PH 7.85 with a concentration of 0.88 mg/mL.

FIG. 2 illustrates the vitreous humor concentration of the compound Formula (I) (“Comp-A”) after intravitreal injection of a nanoparticle formulation (Comp-A-NP-DXA-13) comprising Comp-A.

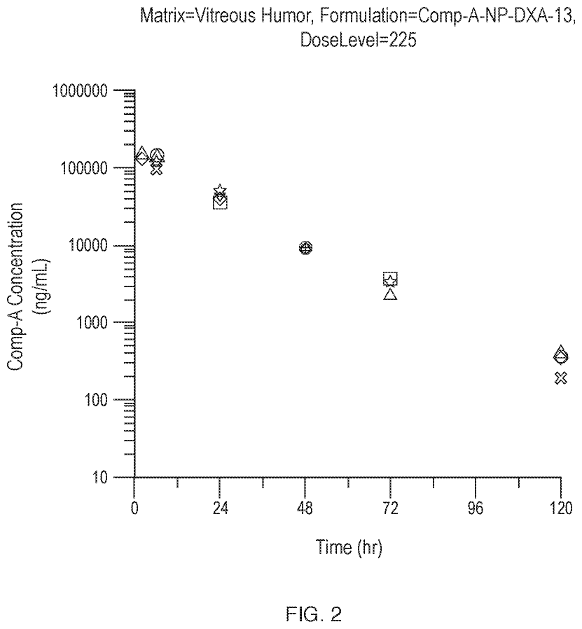

FIG. 3 illustrates the vitreous humor concentration of the compound Formula (I) (“Comp-A”) after intravitreal injection of a nanoparticle formulation (Comp-A-NP-DXA-17) comprising Comp-A.

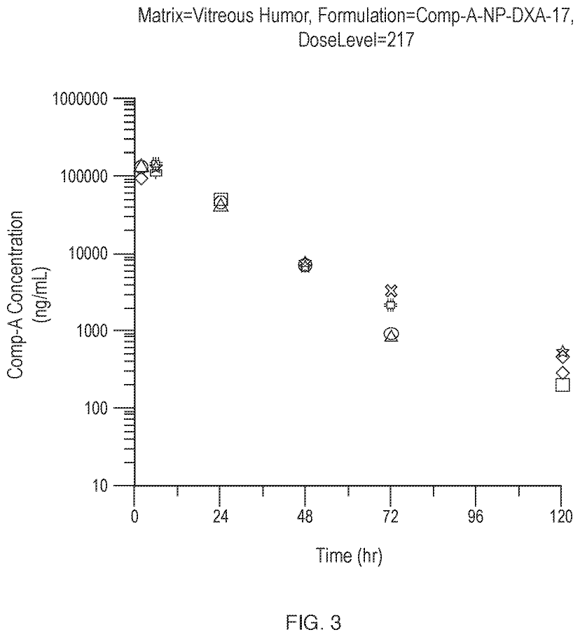

FIG. 4 illustrates the vitreous humor concentration of the compound Formula (I) (“Comp-A”) vs time plots after intravitreal injection of the solution of Comp-A, Comp-A-NP-DXA-13, and Comp-A-NP-DXA-17.

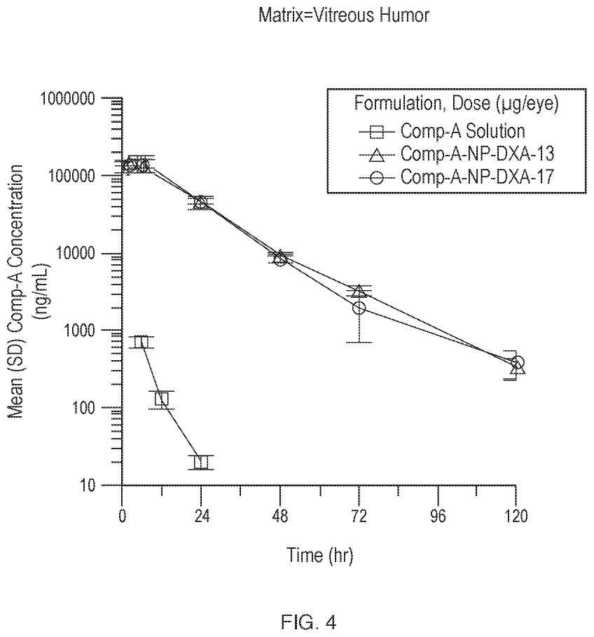

FIG. 5 illustrates the vitreous humor dose-normalized concentration of the compound Formula (I) (“Comp-A”) vs time plots after intravitreal injection of the solution of Comp-A, Comp-A-NP-DXA-13, and Comp-A-NP-DXA-17.

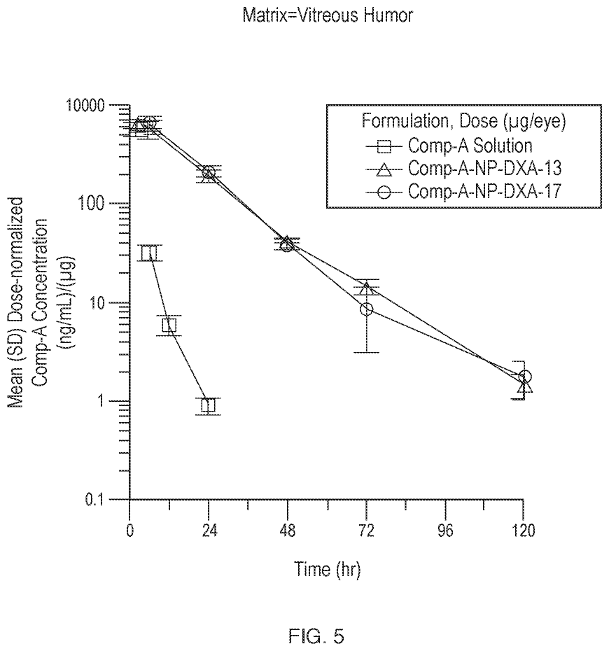

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless defined otherwise, all technical and scientific terms used herein have the same meaning as is commonly understood by one of skill in the art to which this invention belongs.

The terms “co-administration” and “administered in combination with” as used herein, encompass administration of two or more active pharmaceutical ingredients to a subject so that both agents and/or their metabolites are present in the subject at the same time. Co-administration includes simultaneous administration in separate compositions, administration at different times in separate compositions, or administration in a composition in which two or more agents are present.

The term “IC₅₀” refers to the half maximal inhibitory concentration, i.e. inhibition of 50% of the desired activity. The term “EC₅₀” refers to the drug concentration at which one-half the maximum response is achieved.

The term “effective amount” or “therapeutically effective amount” refers to that amount of an active pharmaceutical ingredient or combination of active pharmaceutical ingredients as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, and other factors which can readily be determined by one of ordinary skill in the art. The term also applies to a dose that will induce a particular response in target cells, (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” means quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quaterin die, four times a day, or four times daily.

The term “Polydispersity Index (PDI)” is defined as the square of the ratio of standard deviation (a) of the particle diameter distribution divided by the mean particle diameter (2a), as illustrated by the formula: PDI=(a/2a)². PDI is used to estimate the degree of non-uniformity of a size distribution of nanoparticles, and larger PDI values correspond to a larger size distribution in the particle sample. PDI can also indicate particle aggregation along with the consistency and efficiency of particle surface modifications. A sample is considered monodisperse when the PDI value is less than 0.1.

The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In selected embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve proton transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic, and absorption delaying agents. The use of such media and agents for active pharmaceutical ingredients is well known in the art. Except insofar as any conventional media or agent is incompatible with the active pharmaceutical ingredient, its use in the therapeutic compositions of the invention is contemplated. Supplementary active ingredients can also be incorporated into the described compositions.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

As used herein, the term “ophthalmic condition” or “ophthalmic disorder” includes, but is not limited to, maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

As used herein, the term “macular degeneration” is intended to encompass all forms of macular degeneration and includes a gradual loss of central vision usually affecting either or both eyes that occurs especially in the elderly. A slowly progressing form of macular degeneration, usually referred to as the dry form, is marked especially by the accumulation of yellow deposits in the macula lutea and the thinning of the macula lutea. A rapidly progressing form of macular degeneration, usually referred to as the wet form, is marked by scarring produced by bleeding and fluid leakage from new blood vessels formed below the macula lutea. Macular degeneration may exist as either the wet form or the dry form.

“Solvate” refers to a compound in physical association with one or more molecules of a pharmaceutically acceptable solvent.

Compounds of the invention also include crystalline and amorphous forms of the compound of Formula (I) or Formula (II), including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

### Nanoparticle Composition

The present application relates to a pharmaceutical composition comprising nanoparticles comprising: an MDM2 inhibitor, a surfactant, and a pharmaceutically acceptable excipient. In an embodiment, the MDM2 inhibitor is a compound of Formula (I)

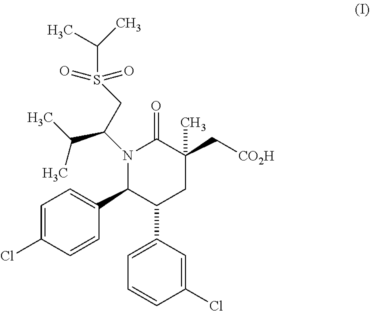 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

or a pharmaceutically acceptable salt thereof.

In an embodiment, the MDM2 inhibitor is a compound of Formula (II)

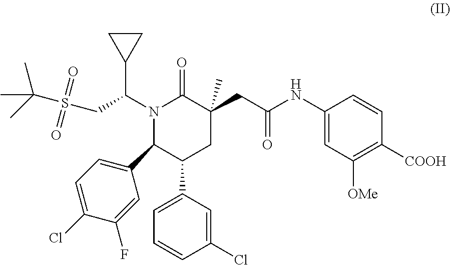 [SMILES: COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In an embodiment, the nanoparticles further comprise a polymer. The polymer is selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In an embodiment, the MDM2 inhibitor is encapsulated in the nanoparticles.

In an embodiment, the polymer is poly(lactic co-glycolic acid) (PLGA).

In an embodiment, PLGA has an average molecular weight of about 10 kDa, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, 140 kDa, or 150 kDa.

In an embodiment, PLGA has lactic acid/glycolic acid ratio of 5:95, 10:90; 15:85; 20:80, 25:75, 30:70; 35:65, 40:60, 45:55, 50:50, 55:45, 60:40, 65:35, 70:30, 75:25, 80:20, 85:15, 80:19, or 95:5.

In an embodiment, the surfactant is selected from the group consisting of polysorbatee, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

In an embodiment, the surfactant is tocopheryl polyethylene glycol succinate (TPGS).

In an embodiment, the nanoparticles further comprise a stabilizer selected from the group consisting of PVP (Povidone), PVA (Polyvinyl alcohol), PEG (Polyethylene glycol), HPMC (Hypromellose), HPC (Hydroxypropyl cellulose), HEC (Hydroxyethyl cellulose), NaCMC (Carboxymethylcellulose sodium), SD (Docusate sodium), SLS (Sodium lauryl sulfate), PEI (Polyehtylene imine), TPGS (D-α-tocopheryl polyethylene glycol succinate), PEO (Polyethylene oxide) and PPO (Polypropylene oxide).

In an embodiment, the nanoparticles further comprise a hydrogel.

In an embodiment, the hydrogel is selected from the group consisting of poly(propylene oxide), poly(ethylene oxide), poloxamers (pluronics), chitosan, gelatin, cellulose derivatives, glycol chitin, poly(N-isopropylacrylamide (PNIPAAm), PEG-PLGA-PEG, [poly(D, L-lactide)-poly(ethyleneglycol)-poly(D,L-lactide) (PDLLA-PEG-PDLLA), and combinations thereof.

### Particle Size and Morphology

In some embodiments, the nanoparticles may have a spherical shape. In some embodiments, the nanoparticles may have cylindrical shape.

In some embodiments, the nanoparticles may have a wide variety of non-spherical shapes. The non-spherical shaped nanoparticles can be used to alter uptake by phagocytic cells and thereby clearance by the reticuloendothelial system. In some embodiments, the non-spherical nanoparticles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

In some embodiments, the particle has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the particle has a median particle size selected from the group consisting of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the nanoparticle has a median particle size of 500 nm. In some embodiments, the nanoparticle has a median particle size of 250 nm.

In an embodiment, the nanoparticles have a median particle size about 5 nm, about 10 nm, about 15 nm, about 20 nm, about 25 nm, about 30 nm, about 35 nm, about 40 nm, about 45 nm, about 50 nm, about 55 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, or about 200 nm.

In an embodiment, the nanoparticles have a median particle size less than about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, or about 230 nm.

In an embodiment, the nanoparticles have a median particle size in a range from about 5 nm to about 200 nm, from about 10 nm to about 190 nm, from about 15 nm to about 180 nm, from about 20 nm to about 175 nm, from about 25 nm to about 170 nm, from about 30 nm to about 165 nm, from about 35 nm to about 160 nm, from about 40 nm to about 155 nm, from about 45 nm to about 150 nm, from about 50 nm to about 145 nm, from about 55 nm to about 140 nm, from about 60 nm to about 135 nm, from about 65 nm to about 130 nm, from about 70 nm to about 125 nm, from about 75 nm to about 120 nm, from about 80 nm to about 115 nm, from about 85 nm to about 110 nm, or from about 90 nm to about 100 nm.

In an embodiment, the nanoparticles have a PDI about 0.05, about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95, or about 1.0.

In an embodiment, the nanoparticles have a PDI less than about 0.05, about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95, or about 1.00.

In an embodiment, the nanoparticles have a PDI in a range from about 0.05 to about 1.00, from about 0.06 to about 0.9, from about 0.07 to about 0.8, from about 0.08 to about 0.7, from about 0.09 to about 0.6, or from about 0.1 to about 0.5.

In some embodiments, the nanoparticles have a PDI from about 0.05 to about 0.15, about 0.06 to about 0.14, about 0.07 to about 0.13, about 0.08 to about 0.12, or about 0.09 to about 0.11. In some embodiments, the nanoparticles have a PDI of about 0.05, about 0.06, about 0.07, about 0.08, about 0.09, about 0.10, about 0.11, about 0.12, about 0.13, about 0.14, or about 0.15

### Polymer

In some embodiments, the nanoparticles further comprise a polymer selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

In some embodiments, the polymer is a lipid selected from the group consisting of lipid, polymer-lipid conjugate, carbohydrate-lipid conjugate, peptide-lipid conjugate, protein-lipid conjugate, and combinations thereof. In some embodiments, the lipid may include one or more of the following: phospholipids such as phosphatidylcholines, phosphatidylserines, phosphatidylinositides, phosphatidylethanolamines, phosphatidylglycerols, phosphatidic acids; sphingolipids such as sphingomyelins, ceramides, phytoceramides, cerebrosides; sterols such as cholesterol, desmosterol, lanthosterol, stigmasterol, zymosterol, diosgenin, and combinations thereof.

In some embodiments, the polymer is conjugated with a lipid to form a polymer-lipid conjugate, wherein the polymers conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether.

In some embodiments, the polymer is a carbohydrate-lipid conjugate, wherein the carbohydrate is conjugated to the lipid and may include monosaccharides (glucose, fructose, glyceraldehydes etc.), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectins, chitin, cellulose, or starch.

In one embodiment, the phospholipid is selected from the group consisting of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group consisting of DPPC, MPPC, PEG, DMPC, DMPG, DSPE, DOPC, DOPE, DPPG, DSPC, DSPE-PEG, MSPC, cholesterol, PS, PC, PE, PG, and combinations thereof.

In some embodiments, the lipid is selected from the group consisting of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the polymer is a biocompatible polymer. In some embodiments, the polymer is a biodegradable polymer.

In some embodiments, the polymer is selected from the group consisting of PDMS (poly (dimethyl siloxane) (PDMS)), polydioxanone, poliglecaprone polypropylene, polyvinylidene fluoride, polyethylene terephthalate, polyethylene including ultra-high-molecular-weight polyethylene (UHMWPE), cross-linked UHMWPE, low density polyethylene (LDPE), high density polyethylene (HDPE), polyketones, polystyrene, polyvinyl chloride, poly (meth) acrylamides, polyetheretherketone (PEEK), poly(methyl methacrylate), polyester including poly(lactic acid-co-glycolic acid) (PLGA), polyglycolic acid (PGA), polylactic acid (PLA), polycaprolactone (PCL), poly(trimethylene carbonate), poly (alpha-esters), polyurethanes, poly(allylamine hydrochloride), poly(ester amides), poly (ortho esters), polyanyhydrides, poly (anhydride-co-imide), cross-linked polyanhydrides, pseudo poly(amino acids), poly (alkylcyanoacrylates), polyphosphoesters, polyphosphazenes, chitosan, collagen, gelatin, natural or synthetic poly(amino acids), elastin, elastin-linked polypeptides, albumin, fibrin, polysiloxanes, polycarbosiloxanes, polysilazanes, polyalkoxysiloxanes, polysaccharides, cross-linkable polymers, thermoresponsive polymers, thermo-thinning polymers, thermo-thickening polymers, block co-polymers comprising polyethylene glycol, and combinations thereof.

In some embodiments, the polymer is selected from the group consisting of PGA, PLA, PLGA, polydioxanone, polycaprolactone, and combinations thereof.

In some embodiments, the polymer is present at a weight percentage by the total weight of the nanoparticles selected from the group consisting of about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7. 5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, or about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, and about 99.0 wt. %. In some embodiments, the polymer is present at a weight percentage by the total weight of the nanoparticles in a range from about 1 wt. % to about 99 wt. %, from about 10.0 wt. % to about 95.0 wt. %, from about 50.0 wt. % to about 95.0 wt. %, from about 25.0 wt. % to about 90.0 wt. % or from about 75.0 wt. % to about 90.0 wt. %.

### Hydrogel

In some embodiments, the nanoparticles further comprise a hydrogel selected from the group consisting of poly(propylene oxide), poly(ethylene oxide), poloxamers (pluronics), chitosan, gelatin, cellulose derivatives, glycol chitin, poly(N-isopropylacrylamide (PNIPAAm), PEG-PLGA-PEG, [poly(D, L-lactide)-poly(ethyleneglycol)-poly(D,L-lactide) (PDLLA-PEG-PDLLA), and combinations thereof. In some embodiments, the hydrogel comprises chitosan and glycol chitosan. In some embodiments, the hydrogel comprises glycol chitin. In some embodiments, the hydrogel is an amphiphilic block copolymer comprising at least on hydrophobic polymer block and at least one hydrophilic polymer block. In some embodiments, the amphiphilic block copolymer is PEG-PLGA-PEG or PDLLA-PEG-PDLLA.

### Additives

In some embodiments, the nanoparticles further include thermal stabilizers. Examples of useful thermal stabilizers include phenolic antioxidants such as butylated hydroxytoluene (BHT), 2-t-butylhydroquinone, and 2-t-butylhydroxyanisole.

In some embodiments, the nanoparticles further include a surfactant. In some embodiments, the surfactant may include cationic, amphoteric, and non-ionic surfactants. In some embodiments, the surfactants comprise anionic surfactants selected from the group consisting of fatty acid salts, bile salts, phospholipids, carnitines, ether carboxylates, succinylated monoglycerides, mono/diacetylated tartaric acid esters of mono- and diglycerides, citric acid esters of mono- and diglycerides, sodium oleate, sodium lauryl sulfate, sodium lauryl sarcosinate, sodium dioctyl sulfosuccinate (SDS), sodium cholate, sodium taurocholate, lauroyl carnitine, palmitoyl carnitine, myristoyl carnitine, lactylic esters of fatty acids, and combinations thereof. In some embodiments, anionic surfactants include di-(2-ethylhexyl) sodium sulfosuccinate. In some embodiments, the surfactants are non-ionic surfactants selected from the group consisting of propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters, and combinations thereof. In some embodiments, the surfactant may comprise fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic amphiphilic surfactant polyoxyethylene (20) sorbitan monolaurate (TWEEN® 20) or polyvinyl alcohol that improves the distribution of IR absorbing material in the polymeric carrier. In some embodiments, the surfactant comprises an amphiphilic surfactant if the IR absorbing material is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

In an embodiment, the surfactant is selected from the group consisting of polysorbatee, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

### Methods Of Treating An Ophthalmic Condition

The present invention relates to a method of treating an ophthalmic condition comprising the step of administering to a human in need thereof a Mouse double minute 2 homolog (MDM2) inhibitor, or a pharmaceutically acceptable salt thereof. The ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

In an embodiment, the MDM2 inhibitor is a compound of Formula (I) or a compound of Formula (II):

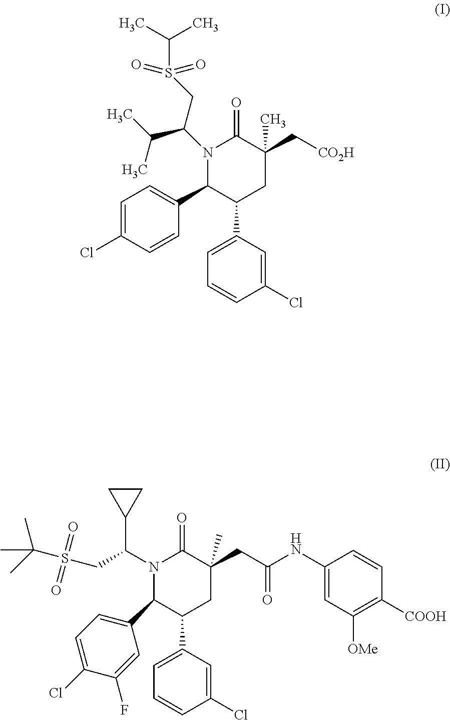 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

or a pharmaceutically acceptable salt thereof.

In an embodiment, the ophthalmic condition in a human subject in need thereof selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof.

In an embodiment, the ophthalmic condition is wet age-related macular degeneration.

In an embodiment, the ophthalmic condition is dry age-related macular degeneration.

In an embodiment, the ophthalmic condition is geographic atrophy (GA). In an embodiment, the geographic atrophy (GA) is multilobular geographic atrophy or unilobular geographic atrophy.

In an embodiment, the ophthalmic condition is macular edema following retinal vein occlusion (RVO).

In an embodiment, the ophthalmic condition is diabetic macular edema (DME).

In an embodiment, the ophthalmic condition is diabetic retinopathy (DR).

In an embodiment, the ophthalmic condition is inflammatory/infectious retinal neovascularization/edema.

In an embodiment, the ophthalmic condition is ocular edema, including post-operative ocular edema.

In an embodiment, the compound of Formula (I) or Formula (II) is in a crystalline form.

In an embodiment, the compound of Formula (I) or Formula (II) is in a free form.

In an embodiment, the MDM2 inhibitor is a pharmaceutically acceptable salt of a compound of Formula (I) or Formula (II).

In an embodiment, the compound of Formula (I) or Formula (II) is in an amorphous form.

In an embodiment, the crystalline form of Formula (I) is characterized by a powder X-ray diffraction pattern comprising at least three peaks at diffraction angle 2 theta degrees selected from a group consisting of peaks at approximately 11.6, 12.4, 18.6, 19.0, 21.6 and 23.6±0.1.

In an embodiment, the human is treated orally with the MDM2 inhibitor on days 1-7 of 21-day cycle, wherein on days 8-21 the human is not treated with the MDM2 inhibitor.

In an embodiment, the compound of Formula (I) or Formula (II) is orally administered.

In an embodiment, the compound of Formula (I) or Formula (II) is topically administered.

In an embodiment, the compound of Formula (I) or Formula (II) is topically administered to the eye.

In an embodiment, the compound of Formula (I) or Formula (II) is administered by intraocular injection to the eye.

In an embodiment, the compound of Formula (I) or Formula (II) is in a dosage form. The dosage form is a solution, suspension, ointment, gel, hydrogel, drug delivery device, tablet, or capsule.

In an embodiment, the therapeutically effective amount of the MDM2 inhibitor is 120 mg.

In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

The present invention also relates to a use of a Mouse double minute 2 homolog (MDM2) inhibitor, or a pharmaceutically acceptable salt thereof, for treating an ophthalmic condition in a human in need thereof, wherein the ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

### Methods Of Treating An Ophthalmic Condition Using Nanoparticles

In another aspect, the present invention relates to a method of treating an ophthalmic condition in a human subject in need thereof selected from the group consisting of wet age-related macular degeneration, dry age-related macular degeneration, diabetic retinopathy (DR), neovascular age-related macular degeneration (nAMD), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, ocular inflammation, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, the method comprising the step of administering to a human subject in need thereof the pharmaceutical composition comprising the nanoparticles described herein by intravitreal injection.

In an embodiment, the ophthalmic condition is neovascular age-related macular degeneration (nAMD) or dry age-related macular degeneration.

In an embodiment, the ophthalmic condition is geographic atrophy (GA).

In an embodiment, the geographic atrophy (GA) is multilobular geographic atrophy or unilobular geographic atrophy.

In an embodiment, the ophthalmic condition is macular edema following retinal vein occlusion (RVO).

In an embodiment, the ophthalmic condition is diabetic macular edema (DME).

In an embodiment, the ophthalmic condition is diabetic retinopathy (DR).

In an embodiment, the ophthalmic condition is inflammatory/infectious retinal neovascularization/edema.

In certain embodiments, the route of delivery used is intraocular injection, direct injection into a given compartment of the eye, such as the vitreous, the cornea, or the retina, application of a patch on the eye, direct application of an ointment, spray, or droppable liquid to the eye, or intraocular implant. In an embodiment, the route of delivery is intravitreal injection.

In an embodiment, the human is treated daily, twice a week, three time a week, weekly, biweekly or monthly.

The present invention also relates to a use of a pharmaceutical composition comprising nanoparticles comprising a Mouse double minute 2 homolog (MDM2) inhibitor, or a pharmaceutically acceptable salt thereof, for treating an ophthalmic condition in a human in need thereof by intravitreal injection, wherein the ophthalmic condition selected from the group consisting of wet age-related macular degeneration, dry age-related macular degeneration, diabetic retinopathy (DR), neovascular age-related macular degeneration (nAMD), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, ocular inflammation, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

### MDM2 Inhibitor

The compound of Formula (I) has the structure and name shown below.

### 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl) Acetic Acid

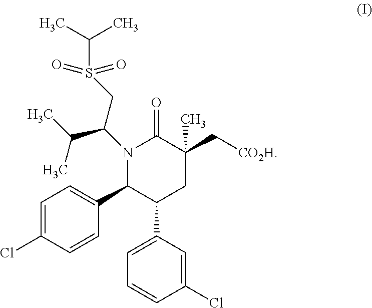 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

The synthesis of the compound of Formula (I) is set forth in International Applications: WO2011/153509 and WO2014/200937; U.S. Pat. Nos. 8,569,341; 9,593,129; 9,296,736; 9,623,018; 9,757,367; 9,801,867; 9; 376; 386; and 9,855,259, the disclosure of which are incorporated by reference herein in its entirety.

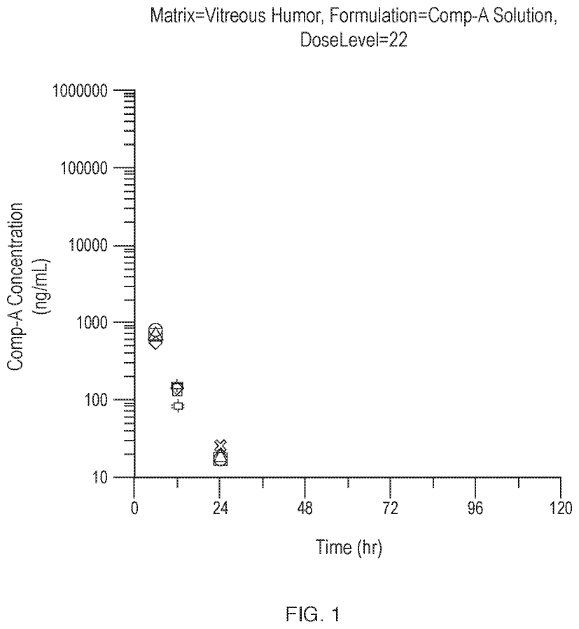

In an embodiment, the compound of Formula (I) is in an amorphous form. In an embodiment, the MDM2 inhibitor is the compound of Formula (I) in a crystalline form. In an embodiment, the MDM2 inhibitor is the compound of Formula (I) in a crystalline anhydrous form. In an embodiment, the MDM2 inhibitor is the compound of Formula (I) in a crystalline anhydrous form characterized by a powder X-ray diffraction pattern comprising peaks at diffraction angle 2 theta degrees at approximately 11.6, 12.4, 18.6, 19.0, 21.6 and 23.6. In an embodiment, the MDM2 inhibitor is the compound of Formula (I) in a crystalline anhydrous form having the X-ray diffraction pattern substantially shown in FIG. 1. The method of making such crystalline form was disclosed in the International Application WO2014200937, the disclosure of which is incorporated herein by reference in its entirety.

In an embodiment, the MDM2 inhibitor is a compound of Formula (II) having the structure and name shown below.

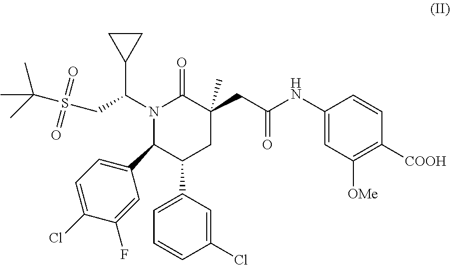 [SMILES: COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]

### 4-(2-((3R,5R,6S)-1-((S)-2-(tert-butylsulfonyl)-1-cyclopropylethyl)-6-(4-chloro-3-fluorophenyl)-5-(3-chlorophenyl)-3-methyl-2-oxopiperidin-3-yl)acetamido)-2-methoxybenzoic Acid

The synthesis of the compound of Formula (II) is set forth in U.S. Pat. No. 8,952,036, the disclosure of which is incorporated by reference herein in its entirety.

### RG7388 (Idasanutlin)

In an embodiment, the MDM2 inhibitor is RG7388. RG7388 has the chemical structure and name shown as:

### 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic Acid

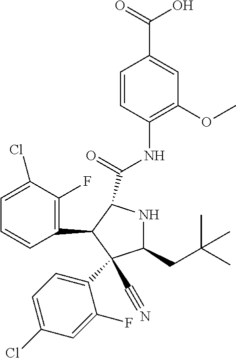 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

### Triptolide (PG490)

In an embodiment, the MDM2 inhibitor is triptolide. Triptolide has the chemical structure and name shown as:

(5bS,6aS,7aS,8R,8aR,9aS,9bS,10aS,10bS)-8-hydroxy-8a-isopropyl-10b-methyl-2,5,5b,6,6a,8,8a,9a,9b,10b-decahydrotris(oxireno) [2′,3′:4b,5; 2″,3″:6,7; 2′″,3′″:8a,9] phenanthro[1,2-c]furan-3 (1H)-one

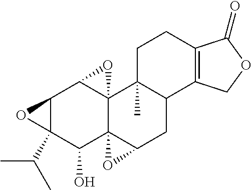

### Nutlin-3a

In an embodiment, the MDM2 inhibitor is Nutlin-3a. Nutlin-3a has the chemical structure and name shown as:

### 4-[(4S,5R)-4,5-bis(4-chlorophenyl)-2-(4-methoxy-2-propan-2-yloxyphenyl)-4,5-dihydroimidazole-1-carbonyl]piperazin-2-one

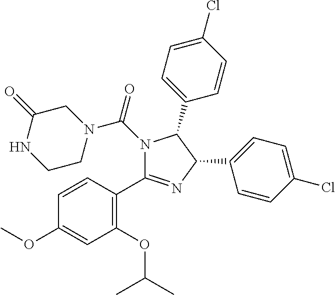 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

### HDM201

In an embodiment, the MDM2 inhibitor is HDM201. HDM201 has the chemical structure and name shown as:

### (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one

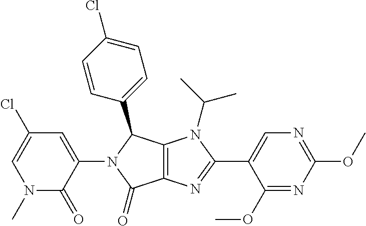 [SMILES: COc1ncc(-c2nc3c(n2C(C)C)[C@H](c2ccc(Cl)cc2)N(c2cc(Cl)cn(C)c2=O)C3=O)c(OC)n1]

### RG7112

In an embodiment, the MDM2 inhibitor is RG7112. RG7112 has the chemical structure and name shown as:

[(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone

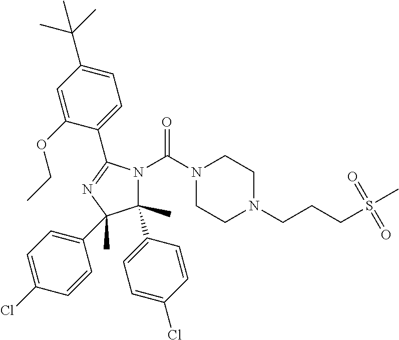 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

### CGM097A

In an embodiment, the MDM2 inhibitor is CGM097A. CGM097A has the chemical structure and name shown as:

### (1S)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one

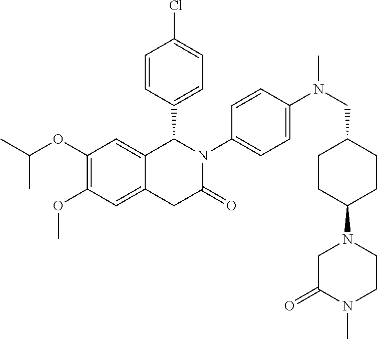 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

### Nutlin-3

In an embodiment, the MDM2 inhibitor is nutlin-3. Nutlin-3 has the chemical structure and name shown as:

### 4-[4,5-bis(4-chlorophenyl)-2-(4-methoxy-2-propan-2-yloxyphenyl)-4,5-dihydroimidazole-1-carbonyl]piperazin-2-one

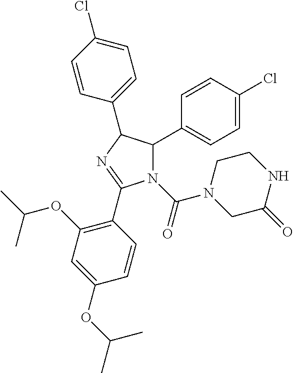 [SMILES: CC(C)Oc1ccc(C2=NC(c3ccc(Cl)cc3)C(c3ccc(Cl)cc3)N2C(=O)N2CCNC(=O)C2)c(OC(C)C)c1]

### SJ-172550

In an embodiment, the MDM2 inhibitor is SJ-172550. SJ-172550 has the chemical structure and name shown as:

### Methyl 2-[2-chloro-6-ethoxy-4-[(3-methyl-5-oxo-1-phenylpyrazol-4-ylidene)methyl]phenoxy]acetate

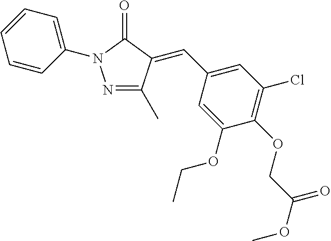 [SMILES: CCOc1cc(/C=C2/C(=O)N(c3ccccc3)N=C2C)cc(Cl)c1OCC(=O)OC]

### SAR405838 (MI-77301)

In an embodiment, the MDM2 inhibitor is SAR405838. SAR405838 has the chemical structure and name shown as:

(2′R,3R,3'S,5'S)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide

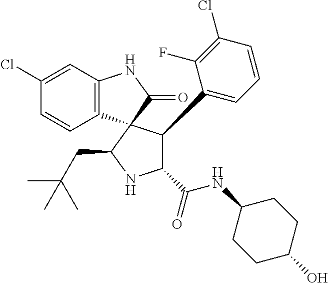 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

### MI-773

In an embodiment, the MDM2 inhibitor is MI-773. MI-773 has the chemical structure and name shown as:

(2′R,3S,3'S,5′R)-6-chloro-3′-(3-chloro-2-fluorophenyl)-5′-(2,2-dimethylpropyl)-N-(4-hydroxycyclohexyl)-2-oxospiro[1H-indole-3,4′-pyrrolidine]-2′-carboxamide

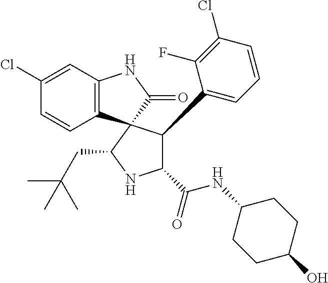 [SMILES: CC(C)(C)C[C@H]1N[C@@H](C(=O)N[C@H]2CC[C@H](O)CC2)[C@H](c2cccc(Cl)c2F)[C@@]12C(=O)Nc1cc(Cl)ccc12]

### MX69

In an embodiment, the MDM2 inhibitor is MX69. MX69 has the chemical structure and name shown as:

### 4-[8-[(3,4-dimethylphenyl)sulfamoyl]-3a,4,5,9b-tetrahydro-3H-cyclopenta[c]quinolin-4-yl]benzoic Acid

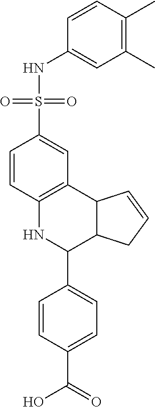 [SMILES: Cc1ccc(NS(=O)(=O)c2ccc3c(c2)C2C=CCC2C(c2ccc(C(=O)O)cc2)N3)cc1C]

### YH239-EE

In an embodiment, the MDM2 inhibitor is YH239-EE. YH239-EE has the chemical structure and name shown as:

### Ethyl 3-[2-(tert-butylamino)-1-[(4-chlorophenyl)methyl-formylamino]-2-oxoethyl]-6-chloro-1H-indole-2-carboxylate

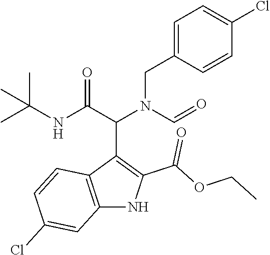 [SMILES: CCOC(=O)c1[nH]c2cc(Cl)ccc2c1C(C(=O)NC(C)(C)C)N(C=O)Cc1ccc(Cl)cc1]

### RO8994

In an embodiment, the MDM2 inhibitor is R08994. R08994 has the chemical structure and name shown as:

(2′R,3R,3′S,5′S)—N-(4-carbamoyl-2-methoxyphenyl)-6-chloro-3′-(3-chloro-2-fluorophenyl)-5′-(2,2-dimethylpropyl)-2-oxospiro[1H-indole-3,4′-pyrrolidine]-2′-carboxamide

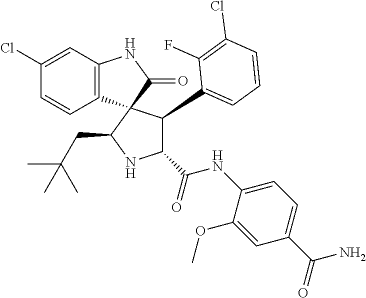 [SMILES: COc1cc(C(N)=O)ccc1NC(=O)[C@@H]1N[C@@H](CC(C)(C)C)[C@@]2(C(=O)Nc3cc(Cl)ccc32)[C@H]1c1cccc(Cl)c1F]

### Nutlin-3b

In an embodiment, the MDM2 inhibitor is nutlin-3b. Nutlin-3b has the chemical structure and name shown as:

### 4-[(4R,5S)-4,5-bis(4-chlorophenyl)-2-(4-methoxy-2-propan-2-yloxyphenyl)-4,5-dihydroimidazole-1-carbonyl]piperazin-2-one

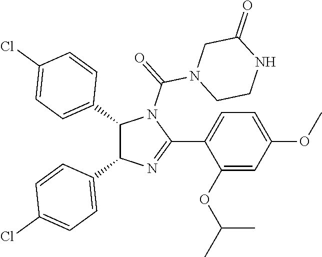 [SMILES: COc1ccc(C2=N[C@H](c3ccc(Cl)cc3)[C@H](c3ccc(Cl)cc3)N2C(=O)N2CCNC(=O)C2)c(OC(C)C)c1]

### Serdemetan (JNJ-26854165)

In an embodiment, the MDM2 inhibitor is Serdemetan. Serdemetan has the chemical structure and name shown as:

### 1-N-[2-(1H-indol-3-yl)ethyl]-4-N-pyridin-4-ylbenzene-1,4-diamine

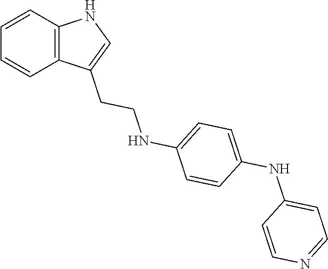 [SMILES: c1ccc2c(CCNc3ccc(Nc4ccncc4)cc3)c[nH]c2c1]

### NSC59984

In an embodiment, the MDM2 inhibitor is NSC59984. NSC59984 has the chemical structure and name shown as:

(E)-1-(4-methylpiperazin-1-yl)-3-(5-nitrofuran-2-yl)prop-2-en-1-one

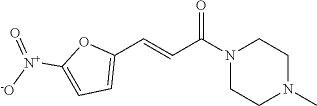

### CHEMBL2386350

In an embodiment, the MDM2 inhibitor is CHEMBL2386350. CHEMBL2386350 has the chemical structure and name shown as:

### 2-[4-[(4S,5R)-2-(4-tert-butyl-2-ethoxyphenyl)-4,5-bis(4-chlorophenyl)-4,5-dimethylimidazole-1-carbonyl]piperazin-1-yl]-1-morpholin-4-ylethanone

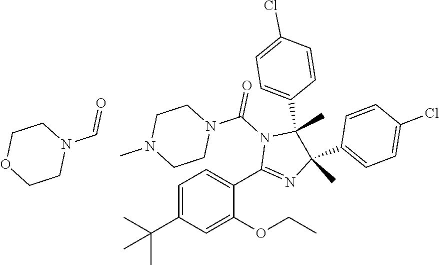

### CGM0970B

In an embodiment, the MDM2 inhibitor is CGM0970B. CGM0970B has the chemical structure and name shown as:

### (1R)-1-(4-chlorophenyl)-6-methoxy-2-[4-[methyl-[[4-(4-methyl-3-oxopiperazin-1-yl)cyclohexyl]methyl]amino]phenyl]-7-propan-2-yloxy-1,4-dihydroisoquinolin-3-one

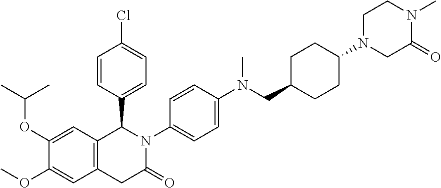

### MK-8242

In an embodiment, the MDM2 inhibitor is MK-8242. MK-8242 has the chemical structure and name shown as:

### 4-amino-1-[(2R,3S,4S,5R)-3,4-dihydroxy-5-(hydroxymethyl)oxolan-2-yl]pyrimidin-2-one

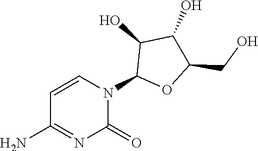 [SMILES: Nc1ccn([C@@H]2O[C@H](CO)[C@@H](O)[C@@H]2O)c(=O)n1]

### DS-3032

In an embodiment, the MDM2 inhibitor is DS-3032. DS-3032 has the chemical structure and name shown as:

(3′R,4'S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide

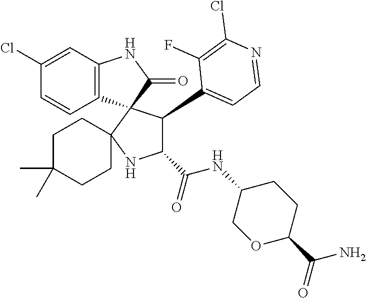 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

### DS-3032B

In an embodiment, the MDM2 inhibitor is DS-3032B. DS-3032B has the chemical structure and name shown as:

(3′R,4′S,5′R)—N-((3R,6S)-6-carbamoyltetrahydro-2H-pyran-3-yl)-6″-chloro-4′-(2-chloro-3-fluoropyridin-4-yl)-4,4-dimethyl-2″-oxodispiro[cyclohexane-1,2′-pyrrolidine-3′,3″-indoline]-5′-carboxamide 4-methylbenzenesulfonate

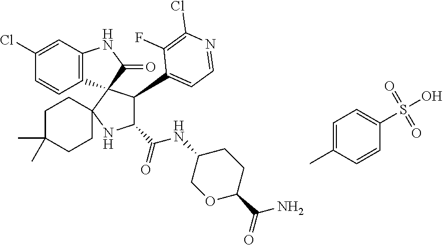 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21 ; Cc1ccc(S(=O)(=O)O)cc1]

### HDM201

In an embodiment, the MDM2 inhibitor is HDM201. HDM201 has the chemical structure and name shown as:

### (4S)-5-(5-chloro-1-methyl-2-oxopyridin-3-yl)-4-(4-chlorophenyl)-2-(2,4-dimethoxypyrimidin-5-yl)-3-propan-2-yl-4H-pyrrolo[3,4-d]imidazol-6-one

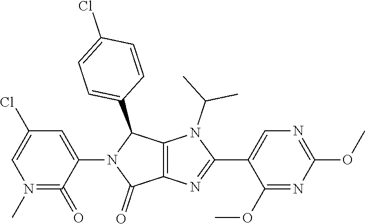

### APG-115

In an embodiment, the MDM2 inhibitor is APG-115. APG-115 has the chemical structure and name shown as:

### 4-((3′R,4'S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido)bicyclo[2.2.2]octane-1-carboxylic Acid

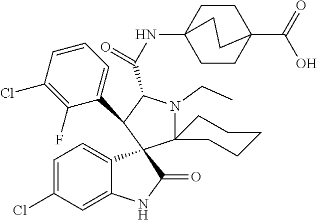 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

### MI-1061

In an embodiment, the MDM2 inhibitor is APG-115. APG-115 has the chemical structure and name shown as:

### 4-((3′R,4'S,5′R)-6″-chloro-4′-(3-chloro-2-fluorophenyl)-2″-oxodispiro[cyclohexane-1,2′-pyrrolidine-3′,3″-indoline]-5′-carboxamido)benzoic Acid

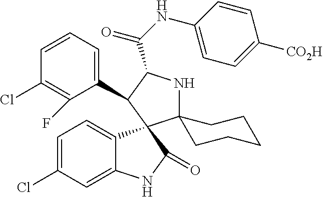 [SMILES: O=C(O)c1ccc(NC(=O)[C@@H]2NC3(CCCCC3)[C@@]3(C(=O)Nc4cc(Cl)ccc43)[C@H]2c2cccc(Cl)c2F)cc1]

### ALRN-6924:

ALRN-6924 is a stapled peptide inhibiting both MDM2 and MDMX from Aileron Therapeutics, and currently in clinical trials (NCT02264613; NCT03725436; NCT04022876; and NCT03654716).

### Pharmaceutical Compositions

In some embodiments, the invention provides pharmaceutical compositions comprising an MDM2 inhibitor or a pharmaceutically acceptable salt thereof for treating an ophthalmic condition, wherein the ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

In an embodiment, the ophthalmic condition selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof.

In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating wet age-related macular degeneration, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating dry age-related macular degeneration, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating geographic atrophy (GA), wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating macular edema following retinal vein occlusion (RVO), wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating diabetic macular edema (DME), wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating diabetic retinopathy (DR), wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

In some embodiments, the invention provides pharmaceutical compositions comprising an MDM2 inhibitor or a pharmaceutically acceptable salt thereof for treating inflammatory/infectious retinal neovascularization/edema, wherein the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof.

The pharmaceutical compositions are typically formulated to provide a therapeutically effective amount of an MDM2 inhibitor or a pharmaceutically acceptable salt thereof. Where desired, the pharmaceutical compositions contain a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants. Where desired, other ingredients in addition to an MDM2 inhibitor or a pharmaceutically acceptable salt thereof may be mixed into a preparation or both components may be formulated into separate preparations for use in combination separately or at the same time.

In selected embodiments, the concentration of an MDM2 inhibitor or a pharmaceutically acceptable salt thereof provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of an MDM2 inhibitor or a pharmaceutically acceptable salt thereof provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of an MDM2 inhibitor or a pharmaceutically acceptable salt thereof is independently in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12% or approximately 1% to approximately 10% w/w, w/v or v/v.

In selected embodiments, the concentration of an MDM2 inhibitor or a pharmaceutically acceptable salt thereof is independently in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In selected embodiments, the amount of an MDM2 inhibitor or a pharmaceutically acceptable salt thereof is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of an MDM2 inhibitor or a pharmaceutically acceptable salt thereof is independently more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

A MDM2 inhibitor or a pharmaceutically acceptable salt thereof are effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

The pharmaceutical composition may be in the form of tablets, gelatin capsules, dragees, syrups, suspensions, solutions, powders, granules, emulsions, or suspensions of microspheres or nanospheres or of lipid or polymeric vesicles for controlled release.

Described below are non-limiting exemplary pharmaceutical compositions and methods for preparing the same.

### Pharmaceutical Compositions for Topical Administration

Pharmaceutical compositions for the topical ophthalmic administration of this invention may be formulated in conventional ophthalmologically compatible vehicles, such as, for example, an ointment, cream, suspension, lotion, powder, solution, paste, gel, hydrogel, spray, aerosol or oil.

The formulation may be one of many topical formulation types containing water as the major ingredient, including solutions, gels, hydrogel, creams, sprays and foams. In an embodiment, the formulation be in the form of an aqueous gel. Accordingly, the formulation of the invention for the topical ophthalmic administration may contain a gelling or thickening agent. Any gelling agent that is water-dispersible is suitable for use in the composition of the invention. One preferred gelling agent is hydroxypropylcellulose, such as that sold under the tradename KLUCEL® (Hercules Incorporated, Wilmington, Del., USA). Another preferred gelling agent is hydroxyethylcellulose, such as that sold under the tradename NATROSOL© (Hercules Incorporated). Other suitable gelling agents include carboxyvinyl polymers, also known as carbomers, such as are sold under the tradename CARBOPOL© 934, 940, 941, 980, and 981 (B.F. Goodrich Co., Akron, Ohio, USA), ETD 2020T^{M}, and ULTREZ© (Noveon, Inc., Cleveland, Ohio, USA). Additional suitable gelling agents are polyvinyl alcohol, polyethylene oxides, propylene glycol alginates, methylcellulose, hydroxypropylmethylcellulose and natural polymeric gums such as xanthan, and carrageenan. The concentration of gelling agent in the composition may be varied depending on several factors, including the desired degree of stabilization of the suspension and desired viscosity of the gel composition.

If desired, the formulation of the invention may further include additional pharmaceutically acceptable excipients typically used in formulations and known to those skilled in the art. Such excipients include, for example, humectants, emollients, pH stabilizing agents, preservatives, chelating agents, and anti-oxidants.

The formulation of the invention for the topical ophthalmic administration may be made by any means by which the components of the invention are combined to provide a pharmaceutical formulation. For example, a suspension of benzoyl peroxide may be made by combining water, the water-miscible organic solvent, and benzoyl peroxide. Preferably, the combination is mixed, such as by stirring, sonicating, milling, and/or shaking, to produce a uniform suspension of benzoyl peroxide particles in the water and organic solvent. Additional ingredients, such as a gelling agent and other excipients, may be added either before or after the uniform suspension is obtained.

Gels comprising polymers can swell in water and then interact in such a way as to thicken the water and increase viscosity. Polymers may interact physically, by chain entanglement, or by ionic or hydrophobic/hydrophilic interactions. In each case, the polymers form a matrix that increases the viscosity of the water and allows for (1) physical stabilization and prevention of migration of suspended the MDM2 inhibitor, (2) maintenance of product homogeneity throughout the shelf life, (3) clean, no drip, no mess transfer of the product from the primary package to the skin surface and (4) easy spreading and acceptable aesthetics.

In an embodiment, the composition for the topical ophthalmic administration comprises matrix builder, such as high molecular weight polyvinylpyrrolidones (e.g., Kollidon© 90F), thicking polymers and biopolymers; poloxamers, emulsifiers, stably suspending oils in gels and solubilizers. The composition for the topical ophthalmic administration may have sensory modifiers such as isopropyl myristate. The solubility in an aqueous matrix can be enhanced by the use of water miscible solvents like propylene glycol, polyethylene glycols, triacetin, poloxamers, and low molecular weight polyvinylpyrrolidone.

In an embodiment, the composition for the topical ophthalmic administration comprises the MDM2 inhibitor suspended in a hydrogel. A hydrogel is a colloidal gel formed as a dispersion in water or other aqueous medium. Thus a hydrogel is formed upon formation of a colloid in which a dispersed phase (the polymer) has combined with a continuous phase (i.e. water) to produce a viscous jellylike product; for example, coagulated silicic acid. A hydrogel is a three-dimensional network of hydrophilic polymer chains that are cross-linked through either chemical or physical bonding. Because of the hydrophilic nature of the polymer chains, hydrogels absorb water and swell (unless they have already absorbed their maximum amount of water). The swelling process is the same as the dissolution of non-cross-linked hydrophilic polymers. By definition, water constitutes at least 10% of the total weight (or volume) of a hydrogel.

Examples of hydrogels include synthetic polymers such as polyhydroxy ethyl methacrylate, and chemically or physically cross-linked polyvinyl alcohol, polyacrylamide, poly(N-vinyl pyrolidone), polyethylene oxide, and hydrolysed polyacrylonitrile. Examples of hydrogels which are organic polymers include covalent or ionically cross-linked polysaccharide-based hydrogels such as the polyvalent metal salts of alginate, pectin, carboxymethyl cellulose, heparin, hyaluronate and hydrogels from chitin, chitosan, pullulan, gellan and xanthan. The preferred hydrogels includes a cellulose compound (i.e. hydroxypropylmethylcellulose [HPMC]) and/or a high molecular weight hyaluronic acid (HA).

The pharmaceutical compositions according to the invention the topical ophthalmic administration may also contain inert additives or combinations of these additives, such as wetting agents; mucoadhesive agent; flavor enhancers; preservatives such as para-hydroxybenzoic acid esters; stabilizers; moisture regulators; pH regulators; osmotic pressure modifiers; emulsifiers; UV-A and UV-B screening agents; and antioxidants, such as α-tocopherol, butylhydroxyanisole or butylhydroxytoluene, superoxide dismutase, ubiquinol or certain metal chelating agents.

The formulations for the topical ophthalmic administration, after sterilization, may be packaged, stored and used directly. In an exemplary embodiment, the formulations are in drop form in the manner typically used to apply eye drops. The normal squeeze-type liquid drop application devices are perfectly suited for use in applying the ophthalmic formulations of the invention. In an exemplary embodiment, the formulations are conveniently administered by dropwise addition of the formulations into the affected eye(s) of the user.

The formulations of the present invention for the topical ophthalmic administration containing preservatives are especially advantageous for use in multi-dose containers. Multi-dose containers, as used herein, refer to containers which allow two or more separate applications of the ophthalmic formulation present within the container. Such containers are resealable—i.e., the container cap may be removed for a first application, and then the cap may be replaced onto the container, thereby providing a substantially liquid impermeable seal again. In various exemplary embodiments, an antimicrobial preservative is present in an amount sufficient to reduce microbial concentrations for a period of about 12 hours to about 1 month, such as about 12 hours to about 3 weeks, such as about 12 hours to about 2 weeks, such as about 12 hours to about 1 week, such as about 12 hours to about 3 days, such as about 12 hours to about 48 hours, such as about 12 hours to about 24 hours.

In an exemplary embodiment, those formulations containing no preservative are packaged in a unit dose container—i.e., where only a single dose can be provided by a given container. Such preservative-free compositions are subject to uncontrolled microbial growth once the consumer initially breaks the container seal. Accordingly, the consumer is instructed to dispose of the container after the first dose. An appropriate unit-dose system such as blow-fill-seal unit dose preservative-free packaging system is typically used for the preservative-free formulations.

The concentration of the MDM2 inhibitor pharmaceutically acceptable salts for the topical ophthalmic administration is typically be about 0.01% to about 10.0% by weight, about 0.02% to about 9.0% by weight, about 0.03% to about 8.0% by weight, about 0.04% to about 7.0% by weight, about 0.05% to about 8.0% by weight, about 0.06% to about 7.0% by weight, about 0.07% to about 6.0% by weight, about 0.08% to about 5.0% by weight, about 0.09% to about 4.0% by weight, about 0.1% to about 3.0% by weight, about 0.2% to about 2.0% by weight, about 0.3% to about 1.0% by weight, about 0.4% to about 5.0% by weight, or about 0.5% to about 5.0% by weight.

The concentration of the MDM2 inhibitor pharmaceutically acceptable salts for the topical ophthalmic administration is typically be about 0.01% by weight, about 0.01% by weight, about 0.02% by weight, about 0.03% by weight, about 0.04% by weight, about 0.05% by weight, about 0.06% by weight, about 0.07% by weight, about 0.08% by weight, about 0.9% by weight, about 0.10% by weight, about 0.15% by weight, about 0.20% by weight, about 0.25% by weight, about 0.30% by weight, about 0.35% by weight, about 0.40% by weight, about 0.45% by weight, about 0.50% by weight, about 0.55% by weight, about 0.6% by weight, about 0.65% by weight, about 0.7% by weight, about 0.75% by weight, about 0.8% by weight, about 0.85% by weight, about 0.9% by weight, about 0.95% by weight, about 1% by weight, about 2% by weight, about 3% by weight, about 4% by weight, about 5% by weight, about 6% by weight, about 7% by weight, about 8% by weight, about 9% by weight, or about 10% by weight.

In various exemplary embodiments, the MDM2 inhibitor or pharmaceutically acceptable salts thereof is employed at a concentration of about 0.1 to about 10% w/v, such as about 0.1 to about 4.5% w/v, such as about 0.1 to about 4.0% w/v, such as about 0.1 to about 3.5% w/v, such as about 0.1 to about 3.0% w/v, such as about 0.1 to about 2.5% w/v, such as about 0.1 to about 2.0% w/v, such as about 0.1 to about 1.5% w/v, such as about 0.1 to about 1.0% w/v, such as about 0.1 to about 0.8% w/v, such as about 0.1 to about 0.7% w/v, such as about 0.1 to about 0.6% w/v, such as about 0.1 to about 0.5% w/v, such as about 0.1 to about 0.4% w/v, such as about 0.1 to about 0.3% w/v, such as about 0.1 to about 0.2% w/v.

Optionally, the formulations for the topical ophthalmic administration contain a tonicity modifier.

In an exemplary embodiment, the tonicity modifier is non-ionic. The tonicity modifier may be selected from, but is not limited to, mannitol, sorbitol, dextrose, sucrose, urea, glycerol, polyethylene glycol and any mixtures thereof. In an exemplary embodiment, the tonicity modifier is present in amount sufficient to generate a tonicity of about 250 to about 350 milliosmoles per kilogram (mOsmol/kg), such as about 265 to about 325 mOsmol/kg, such as about 280 to about 310 mOsmol/kg, such as about 295 to about 315 mOsmol/kg.

The formulation for the topical ophthalmic administration may also contain, an ionic salt, selected from, but not limited to, alkali metal halides (such as, for example, NaCl, KCl, NaBr, etc.), in an amount ranging from about 0.3% to about 1% weight percent or sufficient to approximate the salt concentration and/or tonicity of the human tear fluid. Selected salts from this group may also be referred to as ionic tonicity modifiers.

Where a preservative is used in the formulations for the topical ophthalmic administration, an antimicrobial is present in an amount sufficient to generate a microbial barrier to maintain or reduce microbial concentrations for a period of about 12 hours to about 1 month, such as about 12 hours to about 3 weeks, such as about 12 hours to about 2 weeks, such as about 12 hours to about 1 week, such as about 12 hours to about 3 days, such as about 12 hours to about 48 hours, such as about 12 hours to about 24 hours. Suitable preservatives include, but are not limited to, benzalkonium chloride, benzyl alcohol, sorbic acid, chlorobutanol, cetrimonium, methylparaben, propylparaben, polyamino propyl biguanide, phenylethyl alcohol, chlorhexidine, chlorhexidine digluconate, chloroquat, stabilized oxychloro complex or any combination thereof.

Buffering agents that can be used in the formulations for the topical ophthalmic administration include, but are not limited to, buffers prepared from sodium, potassium bicarbonate, phosphate, acetate, citrate, borate salts and/or phosphoric acid, acetic acid, citric acid or boric acid. In an exemplary embodiment, the buffer is sodium dihydrogen phosphate or disodium phosphate or boric acid/sodium borate. The buffers of the invention should be present in an amount sufficient to produce and maintain a formulation pH of about 5.0 to about 8.0, such as about 5.5 to about 7.7, such as about 6.0 to about 7.5, such as about 6.3 to about 7.5, such as about 6.7 to 7.5, such as about 6.7 to about 7.1, and including a pH of about 5.7, about 5.9, about 6.1, about 6.3, about 6.5, about 6.7, about 6.9, about 7.1, about 7.3, about 7.5, about 7.7 or about 7.9.

A surfactant may also be added to the compositions for the topical ophthalmic administration. In an exemplary embodiment, the surfactant is present at a concentration range of about 0.001% to about 0.3%, such as about 0.005% to about 0.2%, such as about 0.01% to about 0.1%, such as about 0.05% to about 0.1% to provide enhanced wetting characteristics to the formulation. The surfactant may include, but is not limited to, poloxamers, polysorbate 80, polysorbate 20, tyloxapol, polyoxethylene, Brij 35, Brij 58, Brij 78, Aptet 100, G 1045, Spans 20, 40 and 85, Tweens 20, 40, 80 or 81, sodium lauroyl sarcosinate, lauroyl-L-glutamic acid triethanolamine, sodium myristyl sarcosinate and sodium lauryl sulfate, polyoxyethylene sorbitan fatty acid esters, polyoxyethylene hydrogenated castor oil, polyethylene glycol fatty acid esters (e.g., polyoxyl stearate), polyoxyethylene polyoxypropylene alkyl ethers, polyoxyalkylene alkyl phenyl ethers, polyglycerol fatty acids esters (e.g., decaglycerol monolaurate), glycerol fatty acid esters, sorbitan fatty acid esters, and polyoxyethylene polyoxypropylene glycol (poloxamer), polyoxyl stearate 40, and/or any combination thereof.

A stabilizer can also be added to the formulations for the topical ophthalmic administration. Suitable stabilizers include, but are not limited to, sodium metabisulfite, sodium bisulfate, acetylcysteine, ascorbic acid, sodium thiosulfate, alpha-tocopherol, carnosine, retinyl palmitate, salts of ethylenediaminetetraacetic acid (EDTA) (such as, for example, the disodium, tetrasodium, calcium or calcium sodium edetate salts), or any combination thereof.

The mucoadhesive agent, when present in the described formulations, increases corneal contact time, enhances bioavailability and/or produces a lubricating effect, and includes, but is not limited to acrylic acid polymers, methylcellulose, ethylcellulose, Povidone K-30, hydroxypropyl methylcellulose, hydroxyethylcellulose, Carbopol® polymers (such as, for example, Carbopol® 674, 676, 690, 980 NF, EZ-2, EZ-3, EZ-4, Aqua 30 and Novethix™ L-10), hydroxypropyl cellulose, polyvinyl alcohol, gelatin, sodium chondroitin sulfate, or any combination thereof.

In an embodiment, after administration onto the surface of the eye, the composition enters the conjunctiva and anterior sclera and into the corneal layer. When present, the mucoadhesive agent appears to increase residence time in the cornea so that the drug may diffuse slowly over time to the posterior sclera, resulting in delivery of sustained concentrations of the MDM2 inhibitor or pharmaceutically acceptable salts thereof in the posterior sclera. The mucoadhesive agent accomplishes this objective by retarding the loss of the drug through, for example, drainage from the nasolachrimal duct due to lachrymation and tear turnover. The mucoadhesive agent also typically possesses viscosity enhancing properties that may result in a desirable soothing or lubricating effect. The penetration enhancer agent which is optionally added to the formulation enhances penetration of the formulation into the corneal epithelial layers, further enhancing the residence time of the MDM2 inhibitor or pharmaceutically acceptable salts thereof in the eye. The stabilizing agent may act as an antioxidant or otherwise retard the chemical degradation of the MDM2 inhibitor formulation. The buffering agent buffers the formulation to a comfortable near-neutral pH compatible with ocular administration. The tonicity modifier in the formulation produces the appropriate osmolality of the ophthalmic formulation.

The penetration enhancer optionally present in the described formulations for the topical ophthalmic administration includes, but is not limited to, laurocapram (azone), bile acids and their alkali metal salts, including chenodeoxycholic acid, cholic acid, taurocholic acid, taurodeoxycholic acid, tauroursodeoxycholic acid or ursodeoxycholic acid, glycocholate, n-dodecyl-β-D-maltoside, sucrose dodecanoate, octyl maltoside, decyl maltoside, tridecyl maltoside, tetradecyl maltoside, hexamethylene lauramide, hexamethylene octanamide, glycerol monolaurate, PGML (polyethylene glycol monolaurate), dimethyl sulfoxide, methylsulfonylmethane, sodium fusidate, saponins, cyclodextrins (CDs) or any combination thereof.

In addition, a solubilizing or resuspension agent may also be added to the formulations for the topical ophthalmic administration. Suitable solubilizing or resuspension agents include, but are not limited to, cyclodextrins (CDs), such as hydroxypropyl γ-cyclodextrin (Cavasol®), sulfobutyl ether 4 3-cyclodextrin (Captisol®), and hydroxypropyl β-cyclodextrin (Kleptose®) (such as 2-hydroxypropyl 3-cyclodextrin), Polysorbate 80 (Tween80©) or hyaluronic acid or hyaluronate salts. The cyclodextrins in particular may also exhibit penetration enhancing properties, although in other instances, cyclodextrins are known to retard the uptake of steroidal compounds (such as hydrocortisone) into ocular tissues. M. Masson et al., Proc. of the 9^{th} Intl. Symposium on Cyclodextrins, Kluwer Academic Publishers (1999), 363-369; T. Loftsson et al., Acta Ophthalmologica Scandinavica (2003), 144-150; International Journal of Pharmaceutics 156 (1997), 201-209.

An exemplary listing of typical carriers, stabilizers and adjuvants known to those of skill in the art that may be useful in the ophthalmic compositions described herein may be found in Gennaro (2005) Remington: The Science and Practice of Pharmacy, Mack Publishing, 21^{st} ed.

### Pharmaceutical Compositions for Injection

In some embodiments, the invention provides a pharmaceutical composition comprising nanoparticles for injection, wherein the nanoparticles comprise an MDM2 inhibitor or a pharmaceutically acceptable salt thereof, and a pharmaceutical excipient suitable for injection. Components and amounts of agents in the compositions are as described herein.

Aqueous solutions in saline are also conventionally used for injection. Ethanol, glycerol, propylene glycol and liquid polyethylene glycol (and suitable mixtures thereof), and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid and thimerosal.

Sterile injectable solutions are prepared by incorporating an MDM2 inhibitor or a pharmaceutically acceptable salt thereof in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Administration of an MDM2 inhibitor or a pharmaceutically acceptable salt thereof or pharmaceutical composition of these compounds can be effected by any method that enables delivery of the compounds to the site of action. These methods include oral routes, intraduodenal routes, parenteral injection (including intravenous, intra-arterial, subcutaneous, intramuscular, intravascular, intraperitoneal or infusion), topical (e.g., transdermal application), rectal administration, via local delivery by catheter or stent or through inhalation. The combination of compounds can also be administered intraadiposally or intrathecally.

In an embodiment, the pharmaceutical composition comprising an MDM2 inhibitor or a pharmaceutically acceptable salt thereof is administered by intravitreal injection.

In an embodiment, the pharmaceutical composition comprising an MDM2 inhibitor or a pharmaceutically acceptable salt thereof is administered by intraocular injection.

Exemplary parenteral administration forms include solutions or suspensions of active compound in sterile aqueous solutions, for example, aqueous propylene glycol or dextrose solutions. Such dosage forms can be suitably buffered, if desired.

The invention also provides kits. The kits include a pharmaceutical composition comprising nanoparticles comprising an MDM2 inhibitor or a pharmaceutically acceptable salt thereof, either alone or in combination in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer. In an embodiment, the invention provides a kit of a pharmaceutical composition comprising nanoparticles comprising an MDM2 inhibitor or a pharmaceutically acceptable salt thereof for use in the treatment of an ophthalmic condition described herein.

### Dosages and Dosing Regimens

The amount of an MDM2 inhibitor or a pharmaceutically acceptable salt thereof administered will be dependent on the human being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day.

In some embodiments, an MDM2 inhibitor or a pharmaceutically acceptable salt thereof is administered in a single dose. Typically, such administration will be by injection—e.g., intravenous injection or intravitreal injection, in order to introduce the agents quickly. However, other routes may be used as appropriate. A single dose of an MDM2 inhibitor or a pharmaceutically acceptable salt thereof may also be used for treatment of an acute condition.

In some embodiments, an MDM2 inhibitor or a pharmaceutically acceptable salt thereof is administered in multiple doses for treating an ophthalmic condition. In an embodiment, an MDM2 inhibitor or a pharmaceutically acceptable salt thereof is administered in multiple doses. In an embodiment, an MDM2 inhibitor or a pharmaceutically acceptable salt thereof is administered in multiple doses by injection—e.g., intravenous injection or intravitreal injection. In an embodiment, dosing may be once, twice, three times, four times, five times, six times, or more than six times per day. In an embodiment, dosing may be selected from the group consisting of once a day, twice a day, three times a day, four times a day, five times a day, six times a day, once every other day, once weekly, twice weekly, three times weekly, four times weekly, biweekly, and monthly. In other embodiments, an MDM2 inhibitor or a pharmaceutically acceptable salt thereof is administered about once per day to about six times per day. In some embodiments an MDM2 inhibitor or a pharmaceutically acceptable salt thereof is administered once daily, while in other embodiments an MDM2 inhibitor or a pharmaceutically acceptable salt thereof is administered twice daily, and in other embodiments an MDM2 inhibitor or a pharmaceutically acceptable salt thereof is administered three times daily. In some embodiments an MDM2 inhibitor or a pharmaceutically acceptable salt thereof is administered three times a week, including every Monday, Wednesday, and Friday.

In some embodiments, a pharmaceutical composition comprising an MDM2 inhibitor is administered by intravitreal injection to a human subject monthly, bi-monthly, once every three months, quarterly, once every five months, once every six months, or yearly. In some embodiments, the pharmaceutical composition comprising an MDM2 inhibitor is administered by intravitreal or intraocular injection to a human subject monthly for two, three, four, or five months followed by bi-monthly administration.

In some embodiments, a pharmaceutical composition comprising an MDM2 inhibitor is administered topically to a human subject once a day, twice a day, three times a day, once every other day, weekly, twice weekly, three times weekly, four times weekly, biweekly, or monthly.

In some embodiments, a pharmaceutical composition comprising nanoparticles comprising an MDM2 inhibitor is administered by intravitreal or intraocular injection to a human subject monthly, bi-monthly, once every three-month, quarterly, once every five-month, once every six-month, or yearly. In some embodiments, the pharmaceutical composition comprising nanoparticles comprising an MDM2 inhibitor is administered by intravitreal or intraocular injection to a human subject monthly for two, three, four, or five months followed by bi-monthly administration.

In some embodiments, a pharmaceutical composition comprising nanoparticles comprising an MDM2 inhibitor is administered topically to a human subject once a day, twice a day, three times a day, once every other day, weekly, twice weekly, three times weekly, four times weekly, biweekly, or monthly.

Administration of an MDM2 inhibitor or a pharmaceutically acceptable salt thereof may continue as long as necessary. In some embodiments, an MDM2 inhibitor or a pharmaceutically acceptable salt thereof is administered for more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, an MDM2 inhibitor or a pharmaceutically acceptable salt thereof is administered for less than 28, 14, 7, 6, 5, 4, 3, 2, or 1 day. In some embodiments, an MDM2 inhibitor or a pharmaceutically acceptable salt thereof is administered for about 14 days, about 21 days, about 28 days, about 35 days, about 42 days, about 49 days, or about 56 days. In some embodiments, an MDM2 inhibitor or a pharmaceutically acceptable salt thereof is administered chronically on an ongoing basis—e.g., for the treatment of chronic effects. In another embodiment the administration of an MDM2 inhibitor or a pharmaceutically acceptable salt thereof continues for less than about 7 days. In yet another embodiment the administration continues for more than about 6, 10, 14, 28 days, two months, three months, four months, five months, six months, seven months, eight months, nine months, ten months, eleven months or one year. In some embodiments, the administration continues for more than about one year, two years, three years, four years, or five years. In some embodiments, continuous dosing is achieved and maintained as long as necessary.

In some embodiments, an effective dosage of an MDM2 inhibitor or a pharmaceutically acceptable salt thereof is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of an MDM2 inhibitor or a pharmaceutically acceptable salt thereof is about 15 mg, about 25 mg, about 30 mg, about 50 mg, about 50 mg, about 75 mg, about 90 mg, about 100 mg, about 120 mg, about 125 mg, about 150 mg, about 175 mg, about 180 mg, about 200 mg, about 225 mg, about 240 mg, about 250 mg, about 275 mg, about 300 mg, about 325 mg, about 350 mg, about 360 mg, about 375 mg, about 400 mg, about 425 mg, about 450 mg, about 475 mg, about 480 mg, or about 500 mg. In some embodiments, an effective dosage of an MDM2 inhibitor or a pharmaceutically acceptable salt thereof is 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg.

In some embodiments, an effective dosage of an MDM2 inhibitor or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of an MDM2 inhibitor or a pharmaceutically acceptable salt thereof is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some embodiments, an MDM2 inhibitor or a pharmaceutically acceptable salt thereof is administered at a dosage of 10 to 500 mg BID, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg BID.

In some embodiments, an MDM2 inhibitor or a pharmaceutically acceptable salt thereof is administered at a dosage of 10 to 500 mg QD, including a dosage of 15 mg, 25 mg, 30 mg, 50 mg, 60 mg, 75 mg, 90 mg, 100 mg, 120 mg, 150 mg, 175 mg, 180 mg, 200 mg, 225 mg, 240 mg, 250 mg, 275 mg, 300 mg, 325 mg, 350 mg, 360 mg, 375 mg, and 480 mg QD.

An effective amount of an MDM2 inhibitor or a pharmaceutically acceptable salt thereof may be administered in either single or multiple doses by any of the accepted modes of administration of agents having similar utilities, including buccal, sublingual, and transdermal routes, by intra-arterial injection, intravenously, parenterally, intramuscularly, subcutaneously or orally.

In some embodiments, the pharmaceutical composition comprising nanoparticles comprising an MDM2 inhibitor or a pharmaceutically acceptable salt thereof is administered by injection at a dosage of about 0.001 mg/ml, about 0.005 mg/ml, about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5.0 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6.0 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7.0 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8.0 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, or about 50 mg/ml.

In some embodiments, the pharmaceutical composition comprising an MDM2 inhibitor or a pharmaceutically acceptable salt thereof is administered by injection at a volume of about 0.01 ml, about 0.02 ml, about 0.03 ml, about 0.04 ml, about 0.05 ml, about 0.06 ml, about 0.07 ml, about 0.08 ml, about 0.09 ml, about 0.1 ml, about 0.15 ml, about 0.2 ml, about 0.25 ml, about 0.30 ml, about 0.35 ml, about 0.40 ml, about 0.45 ml, about 0.5 ml, about 0.55 ml, about 0.60 ml, about 0.65 ml, about 0.70 ml, about 0.75 ml, about 0.80 ml, about 0.85 ml, about 0.90 ml, about 0.95 ml, about 1.0 ml, about 1.1 ml, about 1.2 ml, about 1.3 ml, about 1.4 ml, about 1.5 ml, about 1.6 ml, about 1.7 ml, about 1.8 ml, about 1.9 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 15.0 ml, about 20.0 ml, about 25.0 ml, about 30.0 ml, about 35.0 ml, about 40.0 ml, about 45.0 ml, or about 50.0 ml.

In some embodiments, the pharmaceutical composition comprising an MDM2 inhibitor or a pharmaceutically acceptable salt thereof is administered by intravitreal injection at a volume of about 0.001 ml, about 0.005 ml, about 0.010 ml, about 0.015 ml, about 0.020 ml, about 0.025 ml, about 0.030 ml, about 0.035 ml, about 0.040 ml, about 0.045 ml, about 0.05 ml, about 0.055 ml, about 0.06 ml, about 0.065 ml, about 0.07 ml, about 0.075 ml, about 0.08 ml, about 0.085 ml, about 0.09 ml, about 0.095 ml, or about 0.1 ml.

In some embodiments, the pharmaceutical composition comprising nanoparticles comprising an MDM2 inhibitor or a pharmaceutically acceptable salt thereof is administered by intravitreal injection at a volume of about 0.001 ml, about 0.005 ml, about 0.010 ml, about 0.015 ml, about 0.020 ml, about 0.025 ml, about 0.030 ml, about 0.035 ml, about 0.040 ml, about 0.045 ml, about 0.05 ml, about 0.055 ml, about 0.06 ml, about 0.065 ml, about 0.07 ml, about 0.075 ml, about 0.08 ml, about 0.085 ml, about 0.09 ml, about 0.095 ml, or about 0.1 ml.

In some embodiments, an MDM2 inhibitor or a pharmaceutically acceptable salt thereof is administered to a subject intermittently, known as intermittent administration. By “intermittent administration”, it is meant a period of administration of a therapeutically effective dose of an MDM2 inhibitor or a pharmaceutically acceptable salt thereof, followed by a time period of discontinuance, which is then followed by another administration period and so on. In each administration period, the dosing frequency can be independently select from three times daily, twice daily, daily, once weekly, twice weekly, three times weekly, four times weekly, five times weekly, or six times weekly for topical administration to the eye or monthly for intravitreal or intraocular injection to the eye.

By “period of discontinuance” or “discontinuance period” or “rest period”, it is meant to the length of time when discontinuing of the administration of an MDM2 inhibitor or a pharmaceutically acceptable salt thereof. The time period of discontinuance may be longer or shorter than the administration period or the same as the administration period. During the discontinuance period, other therapeutic agents other than an MDM2 inhibitor or a pharmaceutically acceptable salt thereof may be administered.

In an embodiment, a pharmaceutical composition comprising nanoparticles comprising an MDM2 inhibitor or a pharmaceutically acceptable salt thereof is administered to a human subject in need thereof by intravitreal or intraocular injection for treating an ophthalmic condition for a first administration period, then followed by a discontinuance period, then followed by a second administration period, and so on, wherein the ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

In an embodiment, a pharmaceutical composition comprising nanoparticles comprising an MDM2 inhibitor or a pharmaceutically acceptable salt thereof is administered topically to a human subject in need thereof for treating an ophthalmic condition for a first administration period, then followed by a discontinuance period, then followed by a second administration period, and so on, wherein the ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, trauma, laser, PDT, photocoagulation, hypoperfusion during surgery, ocular inflammation including post-operative ocular inflammation, radiation retinopathy, bone marrow transplant retinopathy; Proliferative disorders: proliferative vitreal retinopathy and epiretinal membranes, proliferative diabetic retinopathy; Infectious disorders: inflammatory/infectious retinal neovascularization/edema, ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

For topical administration to the eye, the first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which an MDM2 inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period and the second administration period are about one week, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject weekly; and the discontinuance period is about two weeks. In an embodiment, the first administration period and the second administration period are about four weeks, in which an MDM2 inhibitor or a pharmaceutically acceptable salt thereof is administered to a subject daily; and the discontinuance period is about two weeks. In an embodiment, the first administration period and the second administration period are about four weeks, in which an MDM2 inhibitor or a pharmaceutically acceptable salt thereof is administered to a subject weekly; and the discontinuance period is about two weeks. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In an embodiment, a pharmaceutical composition comprising nanoparticles comprising an MDM2 inhibitor or a pharmaceutically acceptable salt thereof is administered to a human subject in need thereof by intravitreal or intraocular injection for treating an ophthalmic condition for a first administration period, then followed by a discontinuance period, then followed by a second administration period, and so on, wherein the ophthalmic condition includes: maculopathies/retinal degeneration: macular degeneration, including age related macular degeneration (AMD), such as wet age related macular degeneration and dry age related macular degeneration, geographic atrophy (GA), choroidal neovascularization, choroidal rupture, retinopathy including diabetic retinopathy, acute and chronic macular neuroretinopathy, central serous chorioretinopathy, retinopathy of prematurity, and macular edema, including cystoid macular edema, and diabetic macular edema; Uveitis/retinitis/choroiditis: acute multifocal placoid pigment epitheliopathy, Behcet's disease, birdshot retinochoroidopathy, uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, rubeosis iridis, and Vogt-Koyanagi-Harada syndrome; Vascular diseases/exudative diseases: retinal vascular occlusive disease, retinal arterial occlusive disease, central retinal vein occlusion, disseminated intravascular coagulopathy, branch retinal vein occlusion, hypertensive fundus changes, ocular ischemic syndrome, ischemic retinopathy, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, proliferative vitreoretinopathy, Eales disease; Traumatic/surgical: sympathetic ophthalmia, uveitic retinal disease, retinal detachment, photocoagulation, hypoperfusion during surgery, ocular inflammation including post-operative ocular inflammation, radiation retinopathy, bone marrow transplant retinopathy; Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum; Retinal tears/holes: retinal detachment, macular hole, giant retinal tear; Tumors: ocular lymphoma, ocular melanoma, ocular tumors, retinal disease associated with tumors, congenital hypertrophy of the RPE, posterior uveitis, posterior uveal melanoma, choroidal hemangioma, choroidal osteoma, choroidal metastasis, choroidal arteriosclerosis, combined hamartoma of the retina and retinal pigmented epithelium, retinoblastoma, vasoproliferative tumors of the ocular fundus, retinal astrocytoma, intraocular lymphoid tumors; Miscellaneous: punctate inner choroidopathy, acute posterior multifocal placoid pigment epitheliopathy, myopic retinal degeneration, myopic neovascularization, pathologic myopia, acute retinal pigment epithelitis, and macular edema following retinal vein occlusion.

The first administration period, the second administration period, and the discontinuance period for intravitreal or intraocular injection are independently selected from the group consisting of one month, two months, three months, four months, five months, six months, seven months, eight months, nice months, ten months, eleven months, and a year, in which the pharmaceutical composition comprising nanoparticles comprising an MDM2 inhibitor or a pharmaceutically acceptable salt thereof is administered to a subject monthly, bi-monthly, once every three-month, once every four-month, once every five-month, once every six-month, or yearly. In an embodiment, the first administration period is at same length as the second administration period. In an embodiment, the first administration period is shorter than the second administration period. In an embodiment, the first administration period is longer than the second administration period. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In an embodiment, the ophthalmic condition is selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof.

In an embodiment, an MDM2 inhibitor or a pharmaceutically acceptable salt thereof is administered to a human subject in need thereof for treating wet age-related macular degeneration for a first administration period, then followed by a discontinuance period, then followed by a second administration period, and so on. The first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which an MDM2 inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period and the second administration period are about one week, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject weekly; and the discontinuance period is about two weeks. In an embodiment, the first administration period and the second administration period are about four weeks, in which an MDM2 inhibitor or a pharmaceutically acceptable salt thereof is administered to a subject daily; and the discontinuance period is about two weeks. In an embodiment, the first administration period and the second administration period are about four weeks, in which an MDM2 inhibitor or a pharmaceutically acceptable salt thereof is administered to a subject weekly; and the discontinuance period is about two weeks. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In an embodiment, an MDM2 inhibitor or a pharmaceutically acceptable salt thereof is administered to a human subject in need thereof for treating geographic atrophy (GA) for a first administration period, then followed by a discontinuance period, then followed by a second administration period, and so on. The first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which an MDM2 inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period and the second administration period are about one week, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject weekly; and the discontinuance period is about two weeks. In an embodiment, the first administration period and the second administration period are about four weeks, in which an MDM2 inhibitor or a pharmaceutically acceptable salt thereof is administered to a subject daily; and the discontinuance period is about two weeks. In an embodiment, the first administration period and the second administration period are about four weeks, in which an MDM2 inhibitor or a pharmaceutically acceptable salt thereof is administered to a subject weekly; and the discontinuance period is about two weeks. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

In an embodiment, an MDM2 inhibitor or a pharmaceutically acceptable salt thereof is administered to a human subject in need thereof for treating age-related macular degeneration for a first administration period, then followed by a discontinuance period, then followed by a second administration period, and so on. The first administration period, the second administration period, and the discontinuance period are independently selected from the group consisting of more than 1, 2, 3, 4, 5, 6, 7, 8, 9, 10, 11, 12, 13, 14, 15, 16, 17, 18, 19, 20, 21, 22, 23, 24, 25, 26, 27, 28, 29, one month, five weeks, six weeks, seven weeks, two months, nine weeks, ten weeks, elven weeks, three months, thirteen weeks, fourteen weeks, fifteen weeks, four months, and more days, in which an MDM2 inhibitor or a pharmaceutically acceptable salt thereof is administered to a subject three times daily, twice daily, daily, once weekly, twice weekly, three times weekly, four times weekly, five times weekly, six times weekly or monthly. In an embodiment, the first administration period is at same length as the second administration period. In an embodiment, the first administration period is shorter than the second administration period. In an embodiment, the first administration period is longer than the second administration period. In an embodiment, the first administration period and the second administration period are about one week, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject daily; and the discontinuance period is about two weeks. In an embodiment, the first administration period and the second administration period are about three weeks, in which an MDM2 inhibitor or a pharmaceutically acceptable salt thereof is administered to a subject weekly; and the discontinuance period is about two weeks. In an embodiment, the first administration period and the second administration period are about four weeks, in which an MDM2 inhibitor or a pharmaceutically acceptable salt thereof is administered to a subject daily; and the discontinuance period is about two weeks. In an embodiment, the first administration period and the second administration period are about four weeks, in which an MDM2 inhibitor or a pharmaceutically acceptable salt thereof is administered to a subject weekly; and the discontinuance period is about two weeks. In an embodiment, the MDM2 inhibitor is the compound of Formula (I) or Formula (II). In an embodiment, the MDM2 inhibitor is selected from the group consisting of a compound of Formula (I), Formula (II), RG7388, Triptolide, HDM201, RG7112, CGM097A, CGM0970B, SJ-172550, SAR405838, MI-773, MX69, YH239-EE, R08994, Nutlin-3, Nutlin-3a, Nutlin-3b, Serdemetan, NSC59984, CHEMBL2386350, MK-8242, DS-3032, DS-3032B, APG-115, MI-1601, ALRN-6924, and pharmaceutically acceptable salts thereof. In an embodiment, the MDM2 inhibitor is selected from the group consisting of a compound of Formula (I), Formula (II), RG7388, HDM201, RG7112, CGM097A, CGM0970B, SAR405838, MK-8242, DS-3032B, APG-115, MI-1601, ALRN-6924, and pharmaceutically acceptable salts thereof.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein. For clarity, the solution of Comp-A refers to the solution of Comp-A in PBS at PH 7.85 with a concentration of 0.88 mg/mL.

### Example 1: Effects of the Compound of Formula (I) or Formula (II) on Endothelial Cells

The procedure of testing the effects of the compound of Formula (I) or Formula (II) on rat retinal microvascular endothelial cell (HRMEC and RRMEC), human umbilical vein endothelial cell (HUVEC), and human umbilical vein SMC (HUVSMC) follows that described in Chavala et al., Journal of Clinical Investigation; 2013; 123(10); 4170-4181, the entirety of which is incorporated by reference. The following describes the procedure briefly.

### Cell Proliferation Assay:

Cells are seeded at a concentration of 2×10⁵ for HUVECs and 2×10⁴ for HUVSMCs in serum-free medium on 12-well plates after detachment. The cells are allowed to adhere overnight in growth medium and then incubated with X-VIVO medium (Cambrex) with or without cytokines (10 ng/ml FGF-2 and 2 μg/ml heparin or 10 ng/ml VEGF-A). For HRMEC experiments, cells are seeded at a concentration of 1×10⁵ and allowed to settle overnight in 6-cm plates coated with attachment factor (Cell Signaling). The next day, fresh 10% FBS and EBM medium will be added. For HUVEC and HUVSMC cell experiments, an equivalent dilution of 100% DMSO, Formula (I) or Formula (II) was added at concentrations indicated in the results the following day. HRMEC experiments were performed in a similar fashion in serum.

Matrigel tube assay for in vitro angiogenesis:

Matrigel matrix (BD) is kept on ice for 24 hours. Then, 200 μl of Matrigel is added to each well of a 24-well culture plate. After hardening the Matrigel at 37° C. for 30 minutes, gels are overlaid with 500 μl of X-VIVO medium containing 3×10⁴ HUVECs. Next, endothelial cells are stimulated with 10 ng/ml of FGF-2 and 2 μg/ml heparin and then incubated with various concentrations of DMSO, Formula (I) or Formula (II), in triplicate, as indicated in the results. The effect of Formula (I) or Formula (II) will be inspected 24 hours under an inverted light microscope. Nine overlapping images with a ×10 objective are taken of each well to perform tubule-length quantification. Adobe Photoshop (Adobe Systems Inc.) is used to make a montage image of each well. Each montage image was then imported into LSM Image Browser v3.5 (Carl Zeiss Inc.) to measure tubule length. Tubule length was then standardized to the overall pixel area examined. A masked observer assessed all measurements.

### Flow Cytometric Analysis for Apoptosis

HUVECs and HUVSMCs are incubated with 7.5 μM of Formula (I) or Formula (II) or DMSO for 24 hours (HUVECs) and 48 hours (HUVSMCs) in X-VIVO medium with FGF-2 (10 ng/ml) and heparin (2 μg/ml). HUVECs are detached with collagenase/EDTA and HUVSMCs are detached with trypsin/EDTA. The cells are then washed twice with PBS and stained with propidium iodide and annexin V-FITC (Annexin V-FITC Apoptosis Detection Kit I, BD) according to the manufacturer's instructions.

### TUNEL Assay for Apoptosis

HUVECs are plated on collagen-coated plastic-bottom culture dishes (MatTek Corp.) overnight. HUVECs are then treated with varying concentrations of Formula (I) or Formula (II) in X-VIVO medium for 24-72 hours. HRMECs are treated with 15 μM of Formula (I) or Formula (II) for 24 hours in 10% FBS. The TUNEL assay is performed following instructions provided with the kit (Roche Applied Science).

### Intravitreal Injection for In Vivo Adult Retinal Vessel Assay

Adult, 3-month-old 129 and C57BL6/129S mice are anesthetized with Avertin and also given a drop of topical proparacaine (1%) for local anesthesia. Using a stereo microscope, a glass capillary pipette was used to inject 1 ml of either vehicle or Formula (I) or Formula (II) into the vitreous cavity of both eyes of each animal. Mice were then euthanized 5 days after injection.

### Subcutaneous and Periocular Injection for In Vivo Retinal Development Assay

A technique described by Strömblad et al. was modified to study the effects of Formula (I) or Formula (II) on in vivo vascular development (J Biol Chem. 2002; 277(16):13371-13374). Briefly, Formula (I) or Formula (II) is administered by subcutaneous injection in the nape or in the periocular area of each eye to WT 129 S1-VIMJ (Jackson Laboratories) mouse pups within 12 hours of birth. The mice will receive a total of 4 (fused eyelids experiments) or 5 (subcutaneous neck experiments) injections of either Formula (I) or Formula (II) or 100% DMSO. The first injection of Formula (I) or Formula (II) is administered at a dose of 40 mg/kg, while the rest are given at a dose of 80 mg/kg for experiments involving injections in the nape. A dose of 80 mg/kg is administered for all injections in the fused eyelid series of experiments. The pups are euthanized on P2 (periocular) or P3 (nape of neck); the eyes are enucleated after the fused eyelids incised.

### Example 2: Formulation for Topical Administration

In this example, various specific topical formulations are illustrated.

### (a) Cream:

| Ingredient | Weight (mg) |
|---|---|
| Compound of Formula (I) or (II) | 3 |
| Carbomer 934 (BF Goodrich Carbopol 974) | 4.5 |
| Disodium edetate | 1 |
| Methyl glucose sesquistearate | 35 |
| PEG methyl glucose sesquistearate | 35 |
| Glycerol | 30 |
| Methyl paraben | 2 |
| Cyclomethicone | 130 |
| Perhydrosqualene | 60 |
| Phenoxyethanol | 5 |
| Propyl paraben | 1 |
| Sodium hydroxide | Adjusting pH to 6.5 ± 0.3 |
| Purified water | q.s. 1 g |

### b) Aqueous Gel

| Ingredient | Weight (mg) |
|---|---|
| Compound of Formula (I) or (II) | 3 |
| Carbomer 940 (BF Goodrich Carbapol 980) | 11 |
| Disodium edetate | 1 |
| Methyl paraben | 2 |
| Poloxamer 124 | 2 |
| Propylene glycol | 40 |
| Sodium hydroxide | Adjusting pH to 6.5 ± 0.3 |
| Purified water | q.s. 1 g |

### Example 3: Pre-Clinical Testing of Topical Administration of Formula (I) or Formula (II)

Male Dutch belted rabbits (n=24) are administered the aqueous gel formulations described herein as 6 eye drops (40 μL) bilaterally, either QD (every 24 hours) or BID (every 12 hours) for 1, 7, and 14 days (n=4/group/dose). Animals are necropsied and ocular tissues are harvested 24 (±2) or 12(±1) hours post last dosing in the QD or BID groups, respectively. Posterior sclera/choroid, aqueous and vitreous humors, and plasma are assayed for the Formula (I) or Formula (II) concentrations using a validated LC-MS/MS method with a lower limit of quantification (LLOQ) of 10 ng/g of tissue. Values below the LLOQ are reported as below quantifiable limits, or BQL. The ocular toxicity and irritation of the formulations will be also evaluated through clinical observations, ocular scoring, intraocular pressure and slit lamp ophthalmoscopy.

### Example 4: Preparation of PLGA Nanoparticles Via Extraction

PLGA nanoparticles were prepared by emulsion technique. Representative nanoparticle formulations (Comp-A-NP-DXA-13 and Comp-A-NP-DXA-17) were made as follows (Comp-A=the compound of Formula (I)):
1. Organic stream: A 20 mg/ml stock of Comp-A and 50 mg/ml stocks of PLGA (Comp-A-NP-DXA-13: 50:50 LA:GA, 0.15-0.25 dl/g, ester terminated; Comp-A-NP-DXA-17: 75/25 LA:GA, 0.55-0.75 dl/g, ester terminated) were made in ACN. Comp-A and PLGA stocks were combined and further diluted in ACN so the final concentrations were 5 mg/ml Comp-A and 10 mg/ml PLGA in ACN.
2. Aqueous stream: A 1% (w/v) stock of d-α-Tocopheryl polyethylene glycol 1000 succinate (TPGS) was made in molecular biology-grade water and acidified to pH 3 with HCl.
3. Dilution buffer: A 10×HBS stock was prepared by dissolving (4-(2-hydroxyethyl)-1-piperazineethanesulfonic acid) (HEPES) and NaCl in Milli-Q water, and adjusting the pH to 7.2. The 10×HBS stock was then sterile filtered and stored at 4° C. Final concentrations of 10×HBS were 0.2 M HEPES, 1.5 M NaCl. Immediately prior to formulation, 10×HBS was diluted 1:10 in Milli-Q water and the pH was adjusted to 7 if necessary to produce 1×HBS (20 mM HEPES and 150 mM NaCl).
4. Polymeric Comp-A formulations were prepared on the NanoAssemblr Benchtop using the formulation scheme described in Table 1.

TABLE 1
Parameters used for formulation of Comp-A
in nanoparticles (NPs) for pharmacokinetic evaluation

| Formulation | Comp-A-NP-DXA-13 | Comp-A-NP-DXA-17 |
|---|---|---|
| PLGA | 50:50 LA:GA, | 75/25 LA:GA, |
| | 0.15-0.25 dl/g, | 0.55-0.75 dl/g, |
| | ester terminated | ester terminated |
| Aqueous stream | 1% TPGS (pH 3) | 1% TPGS (pH 3) |
| Organic stream | 5 mg/ml Comp- | 5 mg/ml Comp- |
| | A + 10 mg/ml | A + 10 mg/ml |
| | PLGA in ACN | PLGA in ACN |
| Flow rate ratio (FRR) | 5:1 | 5:1 |
| (Aqueous TPGS | | |
| to ACN solution) | | |
| Total flow rate (TFR), | 8 | 8 |
| mL/min | | |
| Start waste, mL | 0.45 | 0.45 |
| End waste, mL | 0.05 | 0.05 |
| Dilution buffer | 1XHBS, pH 7 | 1XHBS, pH 7 |

5. Immediately following formulation, NPs were diluted to 4× volume in HBS.
6. Pall 30 kDa MWCO centrifugal filters were used for solvent exchange to HBS and to concentrate formulations (2000×g, 21° C.).
7. Particle size and polydispersity index (PDI) were analyzed using a Malvern Zetasizer (dynamic light scattering; DLS). Comp-A concentrations were determined using UPLC analysis.
8. Final formulations were sterile filtered through 0.22 μm syringe filters and stored at −80° C. in MrFrosty freezing containers. The following day formulations were moved to cryogenic freezer boxes for storage prior to shipping.
9. A sample of each formulation was thawed to room temperature and dialyzed against >1000 volumes of 1×HBS (pH 7) at 37° C. for 6 hours. Samples were collected at 2 mins, 30 mins, 2 hours and 6 hours and analyzed for drug content by UPLC to determine particle release characteristics.

### Results

TABLE 2
Physicochemical characteristics of Comp-A-NP-DXA-13
and Comp-A-NP-DXA-17, and release
kinetics following 1 × freeze/thaw cycle

| | | | | Release at | | |
|---|---|---|---|---|---|---|
| | | | | 37° C.—Remaining | | |
| | Size | | Comp-A | (% of 0 h) | | |
| Formulation | (d.nm) | PDI | (mg/ml) | 30 m | 2 h | 6 h |
| Comp-A-DXA-13 | 98 | 0.194 | 4.505 | 84 | 58 | 45 |
| Comp-A-DXA-17 | 124 | 0.185 | 4.330 | 78 | 62 | 39 |

Comp-A = the compound of Formula (I).

### Example 5: In Vitro Release of Comp-A from the Nanoparticles

Different nanoparticle formulations of Comp-A were test in vitro for the release of Comp-A. The test results are listed in Table 3.

TABLE 3
In vitro release of Comp-A

| | | | | % Release | % Release |
|---|---|---|---|---|---|
| | | | | after | after |
| | Comp-A | size | | 2 h | 6 h |
| Formulation | (mg/mL) | (nm) | PDI | dialysis^{a} | dialysis^{a} |
| Comp-A | | na | na | <1 | 0* |
| unformulated | | | | | |
| PLGA/TPGS | 6.8 | 86 ± 7 | 0.179 ± 0.008 | >75 | 54 ± 11 |
| nanoparticles | | | | | |
| comprising | | | | | |
| Comp-A | | | | | |
| TPGS | 0.7 | 12.6 | 0.171 | 56 | 16** |
| nanoparticles | | | | | |
| comprising | | | | | |
| Comp-A | | | | | |

^{a}Formulations were dialyzed against >1000X volume HBS (pH 7) at 37° C. In addition, unformulated Comp-A was dialyzed as a control
*The control sample (Comp-A solution only) had <3% Comp-A remaining within the dialysis bag after 30 mins
**77% of encapsulated Comp-A remaining within the dialysis bag after 30 minutes
Comp-A = the compound of Formula (I).

### Example 6: In Vivo Release of Comp-A from the Nanoparticles

The total concentration of the compound of Formula (I) (Comp-A) remaining in the rabbit vitreous after intravitreal injection of two different PLGA nanoparticle formulations (Comp-A-NP-DXA-13 and Comp-A-NP-DXA-17) and the solution of Comp-A was investigated.

For this study, Dutch Belted rabbits were received bilateral injections (50 aL/eye) of either Formulation 1 (Comp-A-NP-DXA-13), Formulation 2 (Comp-A-NP-DXA-17). For Formulation 1 and Formulation 2, Comp-A doses were 225 μg/eye and 217 μg/eye, respectively. For the solution of Comp-A dose is 22 μg/eye.

At the indicated timepoints, the animals were euthanized, and the aqueous humor, vitreous humor, iris/ciliary body and retina/choroid snap frozen and individual tissues weighed. Tissues from right and left eyes were collected, weighed, and stored separately. Vitreous samples were processed to release all Comp-A and analyzed using a qualified LC/MS/MS method. Each data point is for a single eye.

TABLE 4
The vitreous humor concentration of Comp-A in different formulations

| | | Time | | Mean | SD |
|---|---|---|---|---|---|
| Matrix | Formulation | (hr) | N | (ng/mL) | (ng/mL) |
| Vitreous Humor | Comp-A Solution | 6 | 4 | 696 | 115 |
| | | 12 | 4 | 129 | 31.1 |
| | | 24 | 4 | 20.1 | 3.83 |
| | | 48 | 0 | BLQ | N/A |
| | | 72 | 0 | BLQ | N/A |
| | | 120 | 0 | BLQ | N/A |
| Vitreous Humor | Comp-A-NP-DXA-13 | 2 | 4 | 140000 | 9540 |
| | | 6 | 4 | 129000 | 25600 |
| | | 24 | 4 | 42900 | 6840 |
| | | 48 | 4 | 9310 | 377 |
| | | 72 | 4 | 3220 | 556 |
| | | 120 | 4 | 330 | 92.2 |
| Vitreous Humor | Comp-A-NP-DXA-17 | 2 | 4 | 122000 | 19900 |
| | | 6 | 4 | 144000 | 24300 |
| | | 24 | 4 | 45400 | 5120 |
| | | 48 | 4 | 8480 | 1080 |
| | | 72 | 4 | 1880 | 1200 |
| | | 120 | 4 | 383 | 157 |

BLQ: below the limit of quantitation of the assay
N/A: not applicable
Comp-A = the compound of Formula (I).

TABLE 5
The vitreous humor dose-normalized concentration of Comp-A in different formulations

| | | Time | | Mean | SD |
|---|---|---|---|---|---|
| Matrix | Formulation | (hr) | N | (ng/mL)/μg | (ng/mL)/μg |
| Vitreous Humor | Comp-A Solution | 6 | 4 | 31.6 | 5.21 |
| | | 12 | 4 | 5.88 | 1.42 |
| | | 24 | 4 | 0.912 | 0.174 |
| | | 48 | 0 | NC | N/A |
| | | 72 | 0 | NC | N/A |
| | | 120 | 0 | NC | N/A |
| Vitreous Humor | Comp-A-NP-DXA-13 | 2 | 4 | 620 | 42.4 |
| | | 6 | 4 | 571 | 114 |
| | | 24 | 4 | 191 | 30.4 |
| | | 48 | 4 | 41.4 | 1.68 |
| | | 72 | 4 | 14.3 | 2.47 |
| | | 120 | 4 | 1.46 | 0.410 |
| Vitreous Humor | Comp-A-NP-DXA-17 | 2 | 4 | 564 | 91.8 |
| | | 6 | 4 | 665 | 112 |
| | | 24 | 4 | 209 | 23.6 |
| | | 48 | 4 | 39.1 | 4.95 |
| | | 72 | 4 | 8.65 | 5.55 |
| | | 120 | 4 | 1.76 | 0.725 |

NC: not calculated (concentration was BLQ in all animals at this timepoint)
N/A: not applicable
Comp-A = the compound of Formula (I).

### Example 7: Tolerability of Intravitreally Administered the Compound of Formula (I)

Based on expected solubility, a maximum feasible intravitreal dose of 44 μg of the compound of Formula (I) in 50 uL pH 7.85 aqueous buffer (sterile saline for injection, phosphate buffered saline or HEPES buffered saline) was administered to Dutch Belted rabbits.

Ophthalmoscopic examinations (slit lamp), ocular scoring, and intraocular pressure were observed through 5 days post-administration.

Results/Conclusion: Intravitreal injection of the solution comprising the compound of Formula (I) produced no ophthalmologic observations through 5 days. The solution produced no findings on clinical or ophthalmologic exam, slit lamp ophthalmoscopy, and no changes in intra-ocular pressure.

The tolerability of 22 microgram/eye and 44 microgram/eye Comp-A in Endotoxin-Free Dulbecco's Phosphate Buffered Saline (PBS) (1×) and in a solution of HEPES buffered saline has been evaluated with no findings.

### Example 8: Pharmacokinetic Evaluation in Dutch Belted Rabbits

Male rabbits were treated with solution comprising the compound of Formula(I) by intraocular injection of 22 μg or 44 μg in sterile PBS, or with nanoparticles comprising the compound of Formula(I), specifically formulations DXA-13 and DXA-17. The concentration of the compound of Formula (I) was measured using a validated LC-MS/MS method in the plasma at various times following intraocular injection. At specified times, a subset of rabbits were euthanized and their eyes dissected for collection of vitreous humor and aqueous humor. The concentrations of the compound of Formula(I) in these tissues were measured using a validated LC-MS/MS method. FIG. 1 shows the concentration-time profile for the compound of Formula (I) in the vitreous humor after a single intraocular injection. The nanoparticle formulations increased the intraocular half-life of the compound of Formula(I), providing an extended exposure, when compared with solution of the same compound. This example demonstrates that in vitro extension of the retention of nanoparticle-encapsulated compound of Formula(I) (Described in Example 5) was predictive of an in vivo extension of exposure following intraocular injection (Table 6).

TABLE 6
Pharmacokinetics data of Comp-A in different formulation

| | | | Formulation | | |
|---|---|---|---|---|---|
| | | | Comp-A Solution | Comp-A-NP-DXA-13 | Comp-A-NP-DXA-17 |
| | | | 22 μg/eye | 225 μg/eye | 217 μg/eye |
| Matrix | Parameter | Units | Estimate | | |
| Vitreous Humor | Tₘₐₓ | hr | 6.00 | 2.00 | 6.00 |
| | Cₘₐₓ | ng/mL | 696 | 140000 | 144000 |
| | AUC₀₋ₜ | hr*ng/mL | 13600 | 2950000 | 2990000 |
| | R² | | 0.974 | 1.00 | 0.970 |
| | λ_{z} | 1/hr | 0.191 | 0.0466 | 0.0416 |
| | t_{1/2} | hr | 3.63 | 14.9 | 16.7 |
| | CL | mL/hr | 1.61 | 0.0761 | 0.0723 |
| | V_{z} | mL | 8.41 | 1.63 | 1.74 |
| | Cₘₐₓ /D | ng/mL/ug | 31.6 | 620 | 665 |
| | AUC₀₋ₜ /D | hr*ng/mL | 618 | 13100 | 13800 |

Comp-A = the compound of Formula (I).

Note: No samples were collected at the 2 hr timepoint in the Comp-A Solution dose group. Therefore, Tₘₐₓ and Cₘₐₓ values relative to the NP dose groups should be interpreted with caution. Additionally, all parameters based on the terminal phase were based on the three available timepoints with measurable concentrations (including Tₘₐₓ). Therefore, these values should be interpreted with caution.

### Example 9: Laser-Induced Choroidal Neovascularization (CNV) Model in Cynomolgus Monkeys

Induction of choroidal neovascularization by inducing laser-injury in the macula of a cynomolgus monkey is a well-characterized animal model of neovascular AMD, which has been used to evaluate effects of treatment for nAMD. For example, the VEGF-targeted therapy aflibercept (Eylea) has been evaluated in such a model. Following the laser injury, the monkeys are monitored for development of choroidal neovascularization by means of fluorescein angiography or indocyanine green angiography, which may be scored qualitatively or further characterized by image analysis to estimate the vascular leak associated with the neovascular lesions. A study of compound in Formula(1) (Comp-A), as a solution formulation or in nanoparticle formulations as described herein, is designed to evaluate the safety and efficacy of intraocular injections in monkeys with laser-induced CNV.

TABLE 7
Study design of CNV model in cynomolgus monkeys

| | Number of | Left Eye | Right Eye | | Animals |
|---|---|---|---|---|---|
| Group | animals | Treatment* | Treatment* | FA | Euthanized |
| Preventative Vehicle | 5 | Vehicle on Days | Vehicle on | Prestudy, | Day 28/29 |
| control & Therapeutic | | 1 & 7 | Day 1 | Days 14 & 28 | |
| aflibercept control | | aflibercept on | aflibercept | | |
| | | Day 14 | on Day 14 | | |
| Comp-A | 5 | 44ug Comp-A on | 44ug Comp-A | Prestudy & | Day 14/15 |
| preventative | | Days 1 & 7 | on Day 1 | Day 14 | |
| Therapeutic Vehicle | 5 | Vehicle on | Vehicle on | Prestudy, | Day 28/29 |
| Control | | Days 14 & 21 | Day 14 | Days 14 & 28 | |
| Comp-A | 5 | 44ug Comp-A on | 44ug Comp-A | Prestudy, | Day 28/29 |
| therapeutic | | Days 14 & 21 | on Day 14 | Days 14 & 28 | |

### Example 9: Choroidal Neovascularization Model in the Dutch Belted Rabbit

Rabbits do not respond to laser-induced retinal injury with a choroidal neovascularization response. Therefore, to evaluate CNV in this species, a model that employs subretinal injections of heparin-sepharose beads with fibroblast growth factor (100 ng) and LPS (100 ng) in 50 μL of ^{˜}3% collagen gel, pH 7.0-7.2. The induction of a chronic model of CNV occurs within 4 weeks (measurable by fluorescein angiography, indocyanine green angiography) and progress of the lesions may also be monitored via fundus photography and spectral domain optical coherence tomography (OCT). This model has advantages over the laser-induced CNV models in rodents and monkeys, as the lesions are more chronic in nature and have an inflammatory phenotype that mimics some aspects of human nAMD. Intraocular injections of the compound of Formula(1), as solution formulated in sterile PBS for injection or HEPES buffered saline, or as a nanoparticle formulation with extended release properties as described in Examples 5 and 7, may evaluated for safety and efficacy after repeated dosing for up to 12 weeks or longer. In this model, a comparator agent such as aflibercept may be injected in a separate group of rabbits to provide a benchmark for the inhibition of VEGF in the treatment of these chronic lesions. The model development and expected results for a VEGF inhibitor are described in Struble et al (2017). Treatment of rabbits with CNV by repeated intraocular injections with the compound of Formula(1), in sterile PBS for injection (pH 7.85) or in a nanoparticle formulation, will be evaluated for safety and efficacy. Based on the intraocular PK studies conducted in Dutch belted rabbits, it will be possible to monitor exposure using plasma PK, allowing estimation of intraocular exposure in rabbits that are treated for 12-24 weeks during this experiment.

TABLE 8
Study design of choroidal neovascularization model in the dutch belted rabbit

| | | | | | Ophthalmic | |
|---|---|---|---|---|---|---|
| | | | | | Exam; Ocular | |
| | Number | CNV | | Comp A | Fluorescein and | Fundus |
| Group | of Animals | Induction^{a} | Treatment | Dose level | ICG Angiography | Photo |
| 1 | 6 | Day 1 | Vehicle | 0 | Weeks 2, 3, 4, 6, | Weeks 2, |
| 2 | 6 | | Comp-A Solution | 44 μg/eye | 8 and 12 | 6 and 12 |
| 3 | 6 | | Comp-A nanoparticle | TBD | | |
| | | | formulation | | | |

^{a}CNV will be induced in 30 animals; form these animals, 18 animals with well-defined CNV lesions will be randomly assigned to Groups 1, 2 or 3 (6 animals/group). CNV will be induced in the right eye only with subretinal injection of 100 ng FGF-2/100 ng LPS/50 μg heparin-sepharose in PBS.
^{b}Beginning on Week 3, 50 μL intravitreal dose in both eyes every 4 weeks for 12 weeks (3 total doses; Weeks 3, 7 & 11); Comp-A will be formulated either as a solution (Group 2) or a nanoparticle formulation (Group 3).

### Example 10: Ocular Tolerability and Estimated Limit Dose of Comp-A-NP-DXA-013 Formulation in Rabbits

Regarding the tolerability dose-response of the PLGA/TPGS nanoparticles; 50 μL of PLGA/TPGS nanoparticle formulations without encapsulated Comp-A (ie, empty particles) at varying polymer concentrations (0.364, 1.09, 3.28, or 9.84 mg/mL) were administered by intravitreal injection to rabbits. The total administered doses were 18, 55, 164 or 492 μg polymer/eye (in 4 eyes) to identify a “limit” dosage of the PLGA/TPGS nanoparticle constituents, as there were no tolerability issues with the Comp-A solution. Ophthalmic examinations, slit lamp examinations and intraocular pressures were conducted approximately 1, 2 and 3 days after administration.
Preliminary Results:
   18 μg/eye—no inflammation noted
   ≥55 μg/eye—dose dependent ocular changes (uveitis)
   At 55 μg/eye—changes resolved by Day 4
   At 162 and 492 μg/eye—changes persisted through Day 4

Based on the empty PLGA/TPGS nanoparticle tolerability results, the formulation can be tolerated in rabbits at 18 μg/eye. At this dosage, the total encapsulated dose of Comp-A in the formulation Comp-A-NP-DXA-013 would be approximately 1.27 μg/eye, assuming the drug:polymer ratio remains consistent. At this dosage, Comp-A-NP-DXA-013 delivers an initial total intravitreal concentration of approximately 1 μg/mL in the vitreous humor of the eye, which would provide target coverage for MDM2 inhibition during an extended period. The long intravitreal half-life (t_{1/2}=14.9 h) of Comp-A-NP-DXA-013 reflects slow clearance (0.0761 mL/hr) due to the release kinetics of Comp-A from the nanoparticle formulation, as demonstrated in Example 8. The duration of MDM2 target coverage can be estimated by comparison to the EC50 for induction of p21 mRNA by Comp-A in the MDM2-amplified tumor cell line SJSA (EC50=26.2 ng/mL). This EC50 is approximately 40 times lower than the total Comp-A concentration in rabbit vitreous humor after the Comp-A-NP-DXA-013 limit dose administration. Given the 14.9 h half-life observed following intravitreous injection of Comp-A-NP-DXA-013, this suggests that target coverage of MDM2 at the rabbit choroid would be maintained for approximately 75 h, at this estimated limit dosage.

### Calculations

The tolerable intravitreal dose of DXA-013 ‘empty’ PLGA/TPGS Nanoparticle formulation in rabbits contained: 18 μg/eye. This amount of DXA-013 PLGA/TPGS NP can encapsulate a total of 1.27 μg of Comp A in a 50 μL injection volume. Equations demonstrating target coverage are shown below.

| | | |
|---|---|---|
| Comp-A-NP-DXA-013 | 0.0706 | 6.86 mg Comp-A/97.1 mg polymer |
| drug: polymer ratio | | |
| Initial Concentration | 1 μg/mL | 18 μg/eye × 0.0706 = 1.27 μg/eye |
| Target EC50 | 26.2 ng/mL | Induction of p21 mRNA in SJSA cells |
| Fold-factor (over | 38.2 × | 1 μg/mL ÷ 0.0262 μg/mL |
| target EC50) | | |
| Half-life of | 14.9 h | See Example 8 |
| Comp-A-NP-DXA-013 | | |
| Approximate time | 75 h | Est. total Comp A after 5 half-lives = 0.031 μg/mL |
| above target EC50 | | |

### Example 11: TPGS Nanoparticle Tolerability

The tolerability of Comp A-loaded TPGS nanoparticles and TPGS nanoparticles without Comp-A encapsulated in Dutch Belted rabbirts was investigated. For this study, 50 μL of Comp A-loaded TPGS nanoparticle formulation (Comp-A, 0.608 mg/mL and TPGS polymer, 11.2 mg/mL; Comp-A, 30 μg/eye and TPGS 560 μg/eye) was injected intravtreally to the right eye of three rabbits; 50 μL of TPGS nanoparticle without Comp-A encapsulated (13.0 mg/mL; 650 μg/eye) was injected intravitrealy into the left eye of the same three rabbits. Ophthalmic examinations and intraocular pressures were conducted approximately 1, 2 and 3 days after administration.
Preliminary Results:
   Comp A-loaded TPGS nanoparticles—uveitis observed on Day 2 and persisted through Day 4.
   TPGS nanoparticles without Comp-A encapsulated—uveitis observed on Day 2 and persisted through Day 4.

1. A method of treating an ophthalmic condition in a human subject in need thereof selected from the group consisting of wet age-related macular degeneration, dry age-related macular degeneration, diabetic retinopathy (DR), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, ocular inflammation, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, the method comprising the step of administering to a human subject in need thereof a therapeutically effective amount of an MDM2 inhibitor, wherein the MDM2 inhibitor is a compound of Formula (I) or a compound of Formula (II): 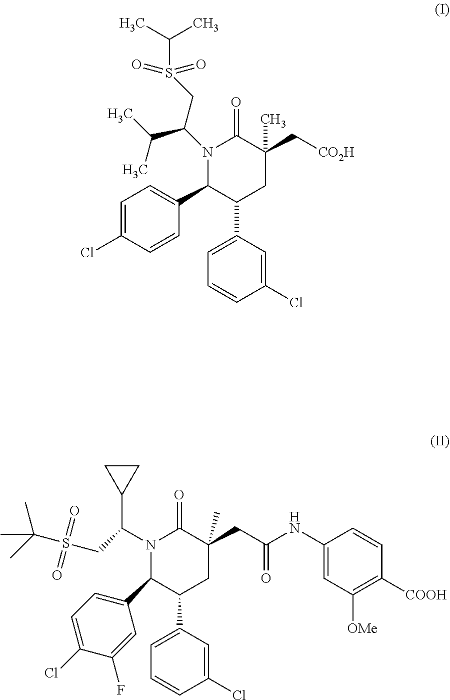 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1 ; COc1cc(NC(=O)C[C@@]2(C)C[C@H](c3cccc(Cl)c3)[C@@H](c3ccc(Cl)c(F)c3)N([C@H](CS(=O)(=O)C(C)(C)C)C3CC3)C2=O)ccc1C(=O)O]
or a pharmaceutically acceptable salt thereof.

2. The method of claim 1, wherein the ophthalmic condition is wet age-related macular degeneration.

3. The method of claim 1, wherein the ophthalmic condition is dry age-related macular degeneration.

4. The method of claim 1, wherein the ophthalmic condition is geographic atrophy (GA).

5. The method of claim 4, wherein the geographic atrophy (GA) is multilobular geographic atrophy or unilobular geographic atrophy.

6. The method of claim 1, wherein the ophthalmic condition is macular edema following retinal vein occlusion (RVO).

7. The method of claim 1, wherein the ophthalmic condition is diabetic macular edema (DME).

8. The method of claim 1, wherein the ophthalmic condition is diabetic retinopathy (DR).

9. The method of claim 1, wherein the ophthalmic condition is inflammatory/infectious retinal neovascularization/edema.

10. The method of claim 1, wherein the compound of Formula (I) or Formula (II) is in a crystalline form.

11. The method of claim 1, wherein the compound of Formula (I) or Formula (II) is in a free form.

12. The method of claim 1, wherein the MDM2 inhibitor is a pharmaceutically acceptable salt of a compound of Formula (I) or Formula (II).

13. The method of claim 1, wherein the compound of Formula (I) or Formula (II) is in an amorphous form.

14. (canceled)

15. The method of claim 1, wherein the human is treated with the MDM2 inhibitor on days 1-7 of 21-day cycle, wherein on days 8-21 the human is not treated with the MDM2 inhibitor.

16. The method of claim 1, wherein the compound of Formula (I) or Formula (II) is intravitreally administered.

17. The method of claim 1, wherein the compound of Formula (I) or Formula (II) is topically administered.

18. The method of claim 1, wherein the compound of Formula (I) or Formula (II) is in a dosage form.

19. The method of claim 18, wherein the dosage form is a solution, suspension, ointment, gel, hydrogel, drug delivery device, tablet, or capsule.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A pharmaceutical composition comprising nanoparticles comprising:
a) an MDM2 inhibitor,
b) a surfactant, and
c) a pharmaceutically acceptable excipient, wherein the MDM2 inhibitor is a compound of Formula (I) 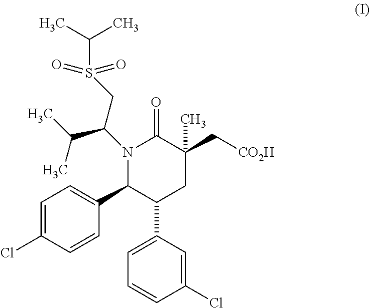 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]
or a pharmaceutically acceptable salt thereof.

40. The pharmaceutical composition of claim 39, wherein the nanoparticles further comprise a polymer.

41. The pharmaceutical composition of claim 39, wherein the nanoparticles have a median particle size less than 150 nm.

42. The pharmaceutical composition of claim 39, wherein the nanoparticles have PDI less than 0.2.

43. The pharmaceutical composition of claim 40, wherein the polymer is selected from the group consisting of chitosan, gelatin, sodium alginate, albumin, poly-L-lactide (PLLA), poly(lactic acid) (PLA), poly(glycolic acid)(PGA), poly(lactic co-glycolic acid) (PLGA), polycaprolactone, poly(lactide co-caprolactone), poly(methyl methacrylates), poloxamer, poly(ethylene glycol) (PEG), PEG-PLLA, PEG-PLGA, poly(methyl vinyl ether/maleic anhydride), cellulose acetate phthalate, and combinations thereof.

44. The pharmaceutical composition of claim 39, wherein the MDM2 inhibitor is encapsulated in the nanoparticles.

45. The pharmaceutical composition of claim 39, wherein the surfactant is selected from the group consisting of polysorbatee, polyvinyl alcohol, methyl cellulose, gelatin, albumin, poloxamer, ethyl cellulose, crosslinked polyacrylic acid polymer, tocopheryl polyethylene glycol succinate (TPGS), sodium cholate, lipids, stearic acid, and combinations thereof.

46. The pharmaceutical nanoparticle composition of claim 39, wherein the surfactant is tocopheryl polyethylene glycol succinate (TPGS).

47. The pharmaceutical composition of claim 40, wherein the polymer is poly(lactic co-glycolic acid) (PLGA).

48. The pharmaceutical composition of claim 43, wherein PLGA has an average molecular weight of about 10 kDa, 30 kDa, or 100 kDa.

49. The pharmaceutical composition of claim 43, wherein PLGA has lactic acid/glycolic acid ratio of 50:50 or 75:25.

50. The pharmaceutical composition of claim 39, wherein the nanoparticles further comprise a hydrogel.

51. The pharmaceutical composition of claim 50, wherein the hydrogel is selected from the group consisting of poly(propylene oxide), poly(ethylene oxide), poloxamers (pluronics), chitosan, gelatin, cellulose derivatives, glycol chitin, poly(N-isopropylacrylamide (PNIPAAm), PEG-PLGA-PEG, [poly(D, L-lactide)-poly(ethyleneglycol)-poly(D,L-lactide) (PDLLA-PEG-PDLLA), and combinations thereof.

52. A method of treating an ophthalmic condition in a human subject in need thereof selected from the group consisting of wet age-related macular degeneration, dry age-related macular degeneration, diabetic retinopathy (DR), neovascular age-related macular degeneration (nAMD), proliferative diabetic retinopathy, ischemic retinopathy, cystoid macular edema, diabetic macular edema (DME), rubeosis iridis, retinopathy of prematurity, retinal vascular occlusive disease, inflammatory/infectious retinal neovascularization/edema, ocular inflammation, retinoblastoma, ocular melanoma, ocular lymphoma, ocular tumors, retinal detachment, myopic neovascularization, angioid streaks, Eales disease, choroidal rupture, proliferative vitreoretinopathy, retinal vein occlusions, sickle cell retinopathy, choroidal hemangioma, choroidal arteriosclerosis, epiretinal membrane, radiation retinopathy, posterior uveitis, pathologic myopia, geographic atrophy (GA), macular edema following retinal vein occlusion, and any combination thereof, the method comprising the step of administering to a human subject in need thereof the pharmaceutical composition of claim 39 by intravitreal injection.

53. The method of claim 52, wherein the ophthalmic condition is neovascular age-related macular degeneration (nAMD) or dry age-related macular degeneration.

54. The method of claim 52, wherein the ophthalmic condition is geographic atrophy (GA).

55. The method of claim 54, wherein the geographic atrophy (GA) is multilobular geographic atrophy or unilobular geographic atrophy.

56. The method of claim 52, wherein the ophthalmic condition is macular edema following retinal vein occlusion (RVO).

57. The method of claim 52, wherein the ophthalmic condition is diabetic macular edema (DME).

58. The method of claim 52, wherein the ophthalmic condition is diabetic retinopathy (DR).

59. The method of claim 52, wherein the ophthalmic condition is inflammatory/infectious retinal neovascularization/edema.

60. The method of claim 52, wherein the MDM2 inhibitor is a pharmaceutically acceptable salt of a compound of Formula (I).

61. The method of claim 52, wherein the human is treated daily, twice a week, three time a week, weekly, biweekly or monthly.

62. (canceled)

63. (canceled)

64. (canceled)

65. (canceled)

66. (canceled)

67. (canceled)

68. (canceled)

69. (canceled)

70. (canceled)

71. (canceled)
